SEQUENCE LISTING (21 sequences)

SEQ ID NO: 1-3 - skipped (3 entries)

SEQ ID NO: 4 - DNA, 60 bp, synthetic construct
  source 1..60: mol_type = other DNA; note = PSP pacDNA
tttttttttttttttnnnnnnnnnnnnnnnnnnnnnnnnnnnnnnttttttttttttttt

SEQ ID NO: 5 - DNA, 30 bp, synthetic construct
  source 1..30: mol_type = other DNA; note = Double PSP pacDNA
nnnnnnnnnnnnnnnttttttttttttttt

SEQ ID NO: 6 - DNA, 46 bp, synthetic construct
  source 1..46: mol_type = other DNA; note = Dumbbell-like PSP pacDNA
nnnnnnnnnnnnnnntttttttttttttttnnnnnnnnnnnnnnnt

SEQ ID NO: 7 - DNA, 15 bp, synthetic construct
  source 1..15: mol_type = other DNA; note = dT15
ttttttttttttttt

SEQ ID NO: 8 - DNA, 15 bp, synthetic construct
  source 1..15: mol_type = other DNA; note = Quencher
aaaaaaaaaaaaaaa

SEQ ID NO: 9 - DNA, 15 bp, synthetic construct
  source 1..15: mol_type = other DNA; note = Dummy quencher
ttttttttttttttt

SEQ ID NO: 10 - DNA, 15 bp, synthetic construct
  source 1..15: mol_type = other DNA; note = HD1
ggttggtgtggttgg

SEQ ID NO: 11 - DNA, 20 bp, synthetic construct
  source 1..20: mol_type = other DNA; note = HD1-DBCO
tttttggttggtgtggttgg

SEQ ID NO: 12 - DNA, 70 bp, synthetic construct
  source 1..70: mol_type = other DNA; note = PSP pacHD1
ggttggtgtggttggtttttnnnnnnnnnnnnnnnnnnnnnnnnnnnnnntttttggttggtgtggttgg

SEQ ID NO: 13 - DNA, 69 bp, synthetic construct
  source 1..69: mol_type = other DNA; note = PSP pacSCR
ggtggtggttgtggttttttnnnnnnnnnnnnnnnnnnnnnnnnnnnnnntttttggtggtggttgtgg

SEQ ID NO: 14 - DNA, 15 bp, synthetic construct
  source 1..15: mol_type = other DNA; note = Antidote
ccaaccacaccaacc

SEQ ID NO: 15 - DNA, 47 bp, synthetic construct
  source 1..47: mol_type = other DNA; note = Cy3-PSP pacDNA
gctattaggagtctttnnnnnnnnnnnnnnnnnnnnnnnnnnnnnnt

SEQ ID NO: 16 - DNA, 16 bp, synthetic construct
  source 1..16: mol_type = other DNA; note = Cy3-oligonucleotide-DBCO
gctattaggagtcttt

SEQ ID NO: 17 - DNA, 16 bp, synthetic construct
  source 1..16: mol_type = other DNA; note = Cy3-oligonucleotide
gctattaggagtcttt

SEQ ID NO: 18 - DNA, 15 bp, synthetic construct
  source 1..15: mol_type = other DNA; note = Cy5-HD1
ggttggtgtggttgg

SEQ ID NO: 19 - DNA, 51 bp, synthetic construct
  source 1..51: mol_type = other DNA; note = Cy5-PSP pacHD1
ggttggtgtggttggtttttnnnnnnnnnnnnnnnnnnnnnnnnnnnnnnt

SEQ ID NO: 20 - DNA, 15 bp, synthetic construct
  source 1..15: mol_type = other DNA; note = Cy5-HD1
ggttggtgtggttgg

SEQ ID NO: 21 - DNA, 50 bp, synthetic construct
  source 1..50: mol_type = other DNA; note = Cy5-PSP pacHD1
nnnnnnnnnnnnnnnnnnnnnnnnnnnnnntttttggttggtgtggttgg

(19) United States
(12) Patent Application Publication
(10) Pub. No.: US 2025/0049935 A1
(43) Pub. Date: Feb. 13, 2025

(54) BOTTLEBRUSH CONJUGATES FOR USE AS OLIGONUCLEOTIDE ENHANCERS

(71) Applicant: Northeastern University, Boston, MA (US)

(72) Inventors: Ke Zhang, Natick, MA (US); Yuyan Wang, Boston, MA (US)

(21) Appl. No.: 18/720,511

(22) PCT Filed: Dec. 16, 2022
(86) PCT No.: PCT/US2022/081857

§ 371 Date: Jun. 14, 2024

Related U.S. Application Data
(60) Provisional application No. 63/290,225, filed on Dec. 16, 2021.

(51) Int. Cl. A61K 47/60; A61K 45/06; C12N 15/115

(52) U.S. Cl. CPC A61K 47/60; A61K 45/06; C12N 15/115; C12N 2310/16

(57) ABSTRACT

Provided herein are, in various embodiments, are bottlebrush polymer-oligonucleotides conjugates. In certain embodiments, the conjugates are potent in vivo, have improved biopharmaceutical properties, enhanced transfection efficiency, and/or an unconventional biodistribution profile. Also provided herein are pharmaceutical compositions, methods of treatment, and methods of making bottlebrush polymer-oligonucleotide conjugates.

### RELATED APPLICATION

This application claims the benefit of U.S. Provisional Application No. 63/290,225, filed on Dec. 16, 2021. The entire teachings of the above application are incorporated herein by reference.

### GOVERNMENT SUPPORT

This invention was made with government support under 1R01CA251730 and 1R01CM121612 from the National Institutes of Health and under 2004947 from the National Science Foundation. The government has certain rights in the invention.

### INCORPORATION BY REFERENCE OF MATERIAL IN XML

This application incorporates by reference the Sequence Listing contained in the following eXtensible Markup Language (XML) file being submitted concurrently herewith:
a) File name: 52001073001.xml; created Dec. 16, 2022, 20,613 Bytes in size.

### BACKGROUND

Oligonucleotides, such as aptamers, face challenges as therapeutics. Aptamers are single-stranded oligonucleotides that can fold into defined secondary structures with a high binding affinity to their targets. Compared to antibodies, aptamers enjoy a wide range of advantages including better production scalability, lower development cost, non-immunogenicity, less susceptibility to biological contamination, better tissue penetration, and greater convenience to develop an antidote. However, these advantages are offset by two serious drawbacks: poor in vivo stability and difficult pharmacological properties (e.g. short blood circulation times, non-specific binding, etc.). It would be helpful to improve and enhance the use of oligonucleotides, including aptamers, to reduce these drawbacks, particularly in a therapeutic setting.

### SUMMARY

In one aspect of the disclosure, there is provided a bottlebrush polymer-oligonucleotide conjugate (conjugate) comprising: a sequence-defined polymer backbone comprising two or more monomers; at least one side chain linked to at least one of the two or more monomers; and at least one oligonucleotide linked to at least one of the two or more monomers. In one aspect of the disclosure, at least one of the monomers is a phosphoramidite, protected amino acid, amino alcohol, amide, monomer comprising a serinol structure, or monomer comprising a pentose structure. In another aspect of the disclosure, the monomers are modified monomers comprising a lipid tail, an aliphatic chain, a cholesterol molecule, a vitamin molecule, a sugar, an amino acid, a peptide, a targeting ligand, an ionizable group, or a combination thereof.

In one aspect of the disclosure, there is provided an arrangement of monomers comprising the sequence-defined polymer backbone that is repeating, non-repeating, symmetrical, asymmetrical, or a combination thereof.

In one aspect of the disclosure, the arrangement of monomers comprising the sequence-defined polymer backbone is selected based on in vitro or in vivo properties of the conjugate. In one aspect of the disclosure, the properties are cellular uptake, subcellular trafficking, pharmacokinetics, biodistribution, toxicity, immunogenicity, transfection efficiency, dry-state diameter, hydrodynamic diameter, zeta potential, or any combination thereof.

In one aspect of the disclosure, each of the monomers comprising the sequence-defined polymer backbone has a defined number of possible side chain conjugation sites and wherein the percentage of side chains covalently linked to the side chain conjugation sites is at least about 80% to at least about 100% per sequence-defined polymer backbone. In one aspect of the disclosure, the at least one side chain is a polysaccharide, a zwitterion polymer, or polyethylene glycol (PEG).

In one aspect of the disclosure, the at least one oligonucleotide is an aptamer, a single-stranded DNA, a double-stranded DNA, a single-stranded RNA, a double stranded RNA, a ribozyme, a DNAzyme, an antisense oligonucleotide, an exon-skipping oligonucleotide, an siRNA oligonucleotide, a triple helix forming oligonucleotide, or any combination thereof.

In one aspect of the disclosure, there is provided a method of treating a disease or disorder, comprising administering to a subject in need thereof, a therapeutically effective amount of a bottlebrush polymer-oligonucleotide conjugate.

In one aspect of the disclosure, the route of administration is intramuscular, intranodal, intravenous, intradermal, subcutaneous, intranasal, infusion, intraperitoneal, intracranial, intratracheal or epicardial.

In one aspect of the disclosure, the disease or disorder affects the subject's lung, ovary, immune system, skin, blood vessel, muscle, blood, brain, heart, intestine(s), pancreas, spleen, kidney, heart, bone, bone marrow, stomach, head, or any combination thereof.

In one aspect of the disclosure, there is provided a method of modulating or altering the expression of a gene product encoded by a target polynucleotide comprising: contacting the target polynucleotide with a bottlebrush polymer-oligonucleotide conjugate.

In one aspect of the disclosure, the efficacy of administration is determined by measuring the subject's plasma pharmacokinetics, blood availability, extrahepatic distribution, tissue retention, dosing frequency or amount, or any combination thereof.

In one aspect of the disclosure, there is provided a method of making a bottlebrush polymer-oligonucleotide conjugate. In one aspect of the disclosure, there is provided a library of bottlebrush polymer-oligonucleotides conjugates.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1A shows schematics of PSP pacDNA synthesis. FIG. 1B shows calibration curve established using glycine as a standard for the TNBSA assay of free primary amines. FIG. 1C shows aqueous GPC chromatograms of PSP pacDNA, intermediate PSP pacDNA after stage 1 PEGylation, PSP backbone, and free dT₁₅. FIG. 1D shows a representative low-magnification AFM image of PSP pacDNA, showing highly homogeneous, non-aggregating spherical particles. FIG. 1E shows the structure of the PSP pacDNA from a coarse-grained molecule dynamics simulation using the MARTINI force field with explicit solvation (dark grey: PEG; light grey: DNA).

FIG. 2A shows ¹H-NMR results for Fmoc-β-Ala-serinol (compound 4). FIG. 2B shows ¹H-NMR results for Fmoc-β-Ala-serinol-DMT (compound 6). FIG. 2C shows ¹H-NMR results for serinol-phosphoramidite (compound 8).

FIG. 3 shows RP-HPLC chromatograms of as-synthesized PSP backbones. The peaks marked with asterisk were fractionated for further reaction.

FIG. 4A shows aqueous GPC chromatograms of the PSP bottlebrushes with DP5, DP20, and DP35. FIG. 4B shows aqueous GPC chromatograms of PSP pacDNAs of various architectures (DNA: dT₁₅). FIG. 4C shows ζ potential measurements of PSP bottlebrushes, PSP pacDNAs, free dT₁₅, and PN pacDNA. FIG. 4D shows representative AFM images of PSP bottlebrushes and PSP pacDNAs. FIG. 4E shows DLS measurements of PSP bottlebrushes (DP5, DP20 and DP35), PN pacDNA, and PSP pacDNAs in Nanopure™ water.

FIG. 5 shows DMF-GPC chromatogram of PSP bottlebrushes and PSP pacDNAs.

FIG. 6A shows TEM images (negatively stained using 2% uranyl acetate) and size distribution measurements of DP20 PSP bottlebrushes. FIG. 6B shows TEM images and size distribution measurements of DP35 PSP bottlebrushes. FIG. 6C shows TEM images and size distribution measurements of PSP pacDNA. FIG. 6D shows TEM images and size distribution measurements of Doubler PSP pacDNA. FIG. 6E shows TEM images and size distribution measurements of Dumbbell-like PSP pacDNA. For FIGS. 6A-E, a minimum of 300 particles per sample were measured.

FIG. 7A shows schematics of hybridization and enzymatic degradation assay. FIG. 7B shows hybridization kinetics of PSP pacDNAs and free dT₁₅. FIG. 7C shows DNase I degradation kinetics of PSP pacDNAs and free dT₁₅.

FIG. 8A shows cellular uptake of PSP pacDNA, PN pacDNA, and free oligonucleotide by HUVEC, NCI-H358, HEP3B, and SKBR3 cells. FIG. 8B shows plasma pharmacokinetics of PSP pacDNA, doubler PSP pacDNA, PSP bottlebrushes, and free dT₁₅. FIG. 8C shows fluorescence imaging of SKH1-Elite mice following i. v. injection of Cy5-labeled PSP bottlebrush with a DP of 30. Statistical significance was calculated using two-way ANOVA. **** P<0.0001.

FIG. 9A shows the structure of PN pacDNA. FIG. 9B shows DMF-GPC chromatogram of PN pacDNA. FIG. 9C is a representative TEM image of PN pacDNA with negative staining (2% uranyl acetate). FIG. 9D shows an aqueous GPC chromatogram of PN pacHD1 and free HD1.

FIG. 10A shows daily fluorescence monitoring of SKH1-Elite mice dosed intravenously with Cy5-labeled PSP bottlebrush (DP30). FIG. 10B shows ex vivo imaging of organs 3, 7, and 37 days post injection. Imaging settings were kept identical.

FIG. 11A shows fluorescence monitoring of athymic mice dosed intravenously with Cy5-labeled PSP bottlebrush (DP30). FIG. 11B shows ex vivo imaging of organs 3 and 14 days post injection. Imaging settings were kept identical.

FIG. 12A shows binding analysis of free HD1, PSP pacHD1, and PSP pacSCR measured by microscale thermophoresis. FIG. 12B shows PT measurements of human plasma treated with samples and controls (5 μM). FIG. 12C shows aPTT measurements of human plasma treated with samples and controls (5 μM). Statistical significance was calculated using Student's two-tailed t test. *** P<0.001, **** P<0.0001.

FIG. 13A shows measurements of PT mouse plasma (ex vivo) treated with samples and controls (5 μM). FIG. 13B shows measurements of aPTT mouse plasma (ex vivo) treated with samples and controls (5 μM). Statistical significance was calculated using Student's two-tailed t test. ** P<0.01, *** P<0.001. FIG. 13C shows plasma pharmacokinetics of free HD1, PSP pacHD1 and PSP bottlebrush (DP30). Statistical significance was calculated using two-way ANOVA. **** P<0.0001. FIG. 13D shows measurements of PT mouse plasma withdrawn at predetermined time points after in vivo injection of samples and controls in C57BL/6 mice. FIG. 13E shows measurements of aPTT mouse plasma withdrawn at predetermined time points after in vivo injection of samples and controls in C57BL/6 mice. FIG. 13F shows tail transection bleeding test of C57BL/6 mice after being treated in vivo with samples and controls. Statistical significance was calculated using Student's two-tailed t test. * P<0.1, *** P<0.001, **** P<0.0001.

FIG. 14A shows plasma pharmacokinetics of 3′- and 5′-Cy5-labeled PSP bottlebrush (DP30-5′-Cy5 and DP30-3′-Cy5) and PSP pacHD1 in C57BL/6 mice. FIG. 14B shows cell viability of NCI-H358 cells treated with HD1 and PSP pacHD1 (0.1 to 5 μM; DNA basis) for 48 h. FIG. 14C shows complement C3 and cytokine levels in the serum of C57BL/6 mice following i.v. injection of PBS, PSP pacHD1, PN pacHD1, free HD1, and lipopolysaccharide (LPS). Statistical significance was calculated using Student's two-tailed t test. **P<0.01, ***P<0.001, ****P<0.0001.

FIG. 15A shows a schematic of pacDNA backbones with varying numbers and arrangement of a lipid-containing modifier. FIG. 15B shows the uptake by NCI-H358 cells of pacDNAs containing varying numbers of lipid tail modifiers evenly distributed across the backbone. FIG. 15C shows the uptake by NCI-H358 cells of pacDNAs containing a fixed number (six) of lipid tail modifiers but with various distribution patterns.

FIG. 16 shows an embodiment of a bottlebrush polymer comprising a sequence-defined polymer backbone comprising two or more monomers, at least one side chain linked to at least one of the two or more monomers, and at least one oligonucleotide linked to at least one of the two or more monomers.

### DETAILED DESCRIPTION

A description of example embodiments follows.

Several aspects of the disclosure are described below, with reference to examples for illustrative purposes only. It should be understood that numerous specific details, relationships, and methods are set forth to provide a full understanding of the disclosure. One having ordinary skill in the relevant art, however, will readily recognize that the disclosure can be practiced without one or more of the specific details or practiced with other methods, protocols, reagents, cell lines, and animals. The present disclosure is not limited by the illustrated ordering of acts or events, as some acts may occur in different orders and/or concurrently with other acts or events. Furthermore, not all illustrated acts, steps, or events are required to implement a methodology in accordance with the present disclosure. Many of the techniques and procedures described, or referenced herein, are well understood and commonly employed using conventional methodology by those skilled in the art.

Unless otherwise defined, all terms of art, notations, and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly-used dictionaries, should be interpreted as having a meaning that is consistent with their meaning in the context of the relevant art and/or as otherwise defined herein.

The terminology used herein is for the purpose of describing particular embodiments only and is not intended to be limiting.

As used herein, the indefinite articles “a,” “an,” and “the” should be understood to include plural reference unless the context clearly indicates otherwise.

Throughout this specification and the claims which follow, unless the context requires otherwise, the word “comprise,” and variations such as “comprises” and “comprising,” will be understood to imply the inclusion of, e.g., a stated integer or step or group of integers or steps, but not the exclusion of any other integer or step or group of integers or steps. When used herein, the term “comprising” can be substituted with the term “containing” or “including.”

As used herein, “consisting of” excludes any element, step, or ingredient not specified in the claim element. When used herein, “consisting essentially of” does not exclude materials or steps that do not materially affect the basic and novel characteristics of the claim. Any of the terms “comprising,” “containing,” “including,” and “having,” whenever used herein in the context of an aspect or embodiment of the disclosure, can in some embodiments, be replaced with the term “consisting of,” or “consisting essentially of” to vary the scope of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and, therefore, satisfy the requirement of the term “and/or” as used herein. Concurrent applicability of more than one of the options is also understood to fall within the meaning, and, therefore, satisfy the requirement of the term “and/or.”

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

Oligonucleotides face challenges as therapeutics. These difficulties are most palpable in vivo due to, e.g., nuclease activities, rapid clearance, and off-target binding. Herein, in an embodiment, the present disclosure demonstrates that a polyphosphodiester-backboned molecular brush can suppress enzymatic digestion, reduce non-specific cell uptake, enable long blood circulation, and rescue the bioactivity of a conjugated oligonucleotide in vivo. The backbone along with the oligonucleotide is assembled via solid-phase synthesis, followed by installation of poly(ethylene glycol) (PEG) side chains using a two-step process with near-quantitative efficiency. The synthesis allows for precise control over polymer size and architecture. Consisting entirely of building blocks that are generally recognized as safe for therapeutics, this novel molecular brush is expected to provide a highly translatable route for oligonucleotide-based therapeutics.

It has been reported that a bottlebrush polymer with dense PEG side chains (e.g., dense PEG side chains) can enhance the plasma pharmacokinetics (PK) and bioavailability of conjugated antisense oligonucleotides by steric inhibition of specific/non-specific nucleic acid-protein interactions (Jia, Fei, et al. “Effect of PEG architecture on the hybridization thermodynamics and protein accessibility of PEGylated oligonucleotides.” Angewandte Chemie 129.5 (2017): 1259-1263; Lu, Xueguang, et al. “Providing oligonucleotides with steric selectivity by brush-polymer-assisted compaction.” Journal of the American Chemical Society 137.39 (2015): 12466-12469; the contents of which are incorporated herein by reference in their entirety). Such steric selectivity greatly reduces side effects associated with oligonucleotide therapeutics, including coagulopathy and unwanted activation of the immune system. Termed pacDNA (polymer-assisted compaction of DNA) or conjugate, these nanoscopic bioconjugates produce a novel biodistribution profile, elevate blood circulation times, and augment tissue retention (up to 15 weeks post intravenous injection). These improvements result in massively boosted antisense activities in vivo, with 1-2 orders of magnitude reduction in dosage requirement in certain cancer xenograft models. However, these initial pacDNAs may not be ideal for certain forms of oligonucleotides, such as aptamers, due to their tendency to undergo endocytosis, which is speculated to stem from the hydrophobic polynorbornene (PN) backbone of the bottlebrush polymer. Thus, a new chemistry that provides variable biological properties for the pacDNA is preferable. The study of the pacDNA backbone sequence and composition and the corresponding biological properties is referred to as “backbonomics.” Herein, in an embodiment, a novel pacDNA with a sequence-defined polyphosphodiester backbones capable of generating a range of biological properties such as plasma pharmacokinetics, biodistribution, and cell uptake, is disclosed.

Aptamers have seen very limited commercial success, with only one drug on the market: Macugen®, a PEGylated, multi-modified aptamer that is delivered locally (intravitreal) to treat age related macular degeneration. Even in this space, Macugen® is facing severe competition from antibody alternatives (Lucentis® or off-label use of Avastin®) that bind to the same target, vascular endothelial growth factor A.

Aptamers that bind to a specific target are selected from a random sequence library by a process termed systematic evolution of ligands by exponential enrichment (SELEX). Advances in SELEX technologies have enabled a small number of chemical modifications such as 2′-fluoro, 2′-amino, and α-nucleoside thiotriphosphates (Sp) to be incorporated into the selection process, which render the resulting aptamers more resistant towards degrading enzymes. Aptamers can also be tested post-SELEX for tolerance of modifications that can further enhance their properties, such as 2′-OMe substitution of purines, 3′-capping, and bioconjugation (e.g. with lipids, cholesterol, or polymers). Further, aptamers have been prepared using the enantiomeric form of natural nucleic acids (Spiegelmer®), which makes such aptamers completely unsusceptible to nucleases. Together, these advances have considerably addressed the in vivo stability aspect of aptamers, leaving pharmacological limitations a primary hurdle for clinical translation.

### Compositions and Methods of the Disclosure

In one aspect, the present disclosure provides for a bottlebrush polymer-oligonucleotide conjugate (conjugate). In some embodiments, the conjugate comprises a sequence-defined polymer backbone comprising two or more monomers; at least one side chain linked to at least one of the two or more monomers; and at least one oligonucleotide linked to at least one of the two or more monomers. In some embodiments of the disclosure, the conjugate is biocompatible.

Also provided herein are methods of making bottlebrush polymer-oligonucleotide conjugates. In some embodiments, method of making comprise polymerizing a sequence-defined polymer backbone comprising two or more monomers. In some embodiments, polymerization is conducted iteratively. In some embodiments, polymerization uses solid-phase synthesis. In other embodiments, polymerization uses solution phase synthesis. In some embodiments, the polymer backbone is made from a mixture of different monomers in each coupling step, to provide a library of randomized backbones. In some embodiments, the backbone is synthesized on a solid support. In some embodiments, the backbone is synthesized in solution. In some embodiments, the backbone is synthesized by iteratively connect the monomers together stepwise, e.g., on a solid support. In some embodiments, the polymer backbone is designed to comprise, i.e., contain, an arbitrary arrangement of different monomers.

In still further embodiments, the compositions and methods disclosed herein provide for conjugates wherein in at least one monomer is a phosphoramidite, protected amino acid, amino alcohol, amide, monomer comprising a serinol structure, monomer comprising a pentose structure. In some embodiments, at least one monomer is a custom phosphoramidite. In some embodiments, the sequence-defined polymer backbone comprises at least one serinol-phosphoramidite monomer. In some embodiments, the at least one of the monomers is a modified monomer. In some embodiments, the modified monomer is a monomer comprising a lipid tail, an aliphatic chain, a cholesterol molecule, a vitamin molecule, a sugar, an amino acid, a peptide, a targeting ligand, an ionizable group, or any combination thereof.

Also provided herein are libraries of randomized backbones (e.g., for use in making the conjugates), and methods of making a library of randomized backbones for use in making bottlebrush polymer-oligonucleotide conjugates, for example, by polymerizing at least two sequence-defined polymer backbones, wherein each sequence-defined polymer backbone comprises two or more monomers, and wherein the sequence of monomers comprising each backbone is different from each other backbone.

In some embodiments, the compositions and methods comprise an arrangement of the monomers in an order. In some embodiments, the arrangement of monomers comprising the sequence-defined polymer backbone is repeating, non-repeating, symmetrical, asymmetrical, or a combination thereof. In some embodiments, the arrangement of monomers is in sets of two, three, four, or five. In some embodiments, the arrangement of monomers is such that modified monomers are flanked by unmodified monomers. In some embodiments, unmodified monomers are flanked by modified monomers. In some embodiments, the arrangement of monomers is selected based on in vitro and/or in vivo properties of the conjugate. In still further embodiments, the in vitro and/or in vivo properties are selected from cellular uptake, subcellular trafficking, pharmacokinetics, biodistribution, toxicity, immunogenicity, transfection efficiency, dry-state diameter, hydrodynamic diameter, zeta potential, and any combination thereof. In some embodiments, the sequence-defined polymer backbone is hydrophilic. In some embodiments, the sequence-defined polymer backbone is hydrophobic. In still further embodiments, the sequence-defined polymer is completely hydrophilic.

In some embodiments, the dry-state diameter of the conjugate is at about 5-30, 5-10, 10-15, 15-25, 20-25, 20-30, or 25-30 nm. In some embodiments, the hydrodynamic diameter of the conjugate is about 10-30, 15-30, 15-25, 20-30, 25-30 nm. In some embodiments, the zeta potential is negative. In some embodiments, the zeta potential is negative is similar to the zeta potential of free oligonucleotides.

In some embodiments, the backbone comprises at least one modifier. In some embodiments, at least one modifier is positively charged. In some embodiments, at least one modifier increases or decreases zeta potential as compared to a conjugate lacking at least one modifier. In some embodiments, the one modifier increases or decreases hydrophobicity compared to a conjugate lacking at least one modifier. In some embodiments, the modifier is spermine, cholesterol, or triphenylalanine. In some embodiments, the backbone comprises at least two or more modifiers. In some embodiments, the modifiers are evenly distributed. In some embodiments, the modifiers have varying grouping densities.

In some embodiments of the disclosure, the conjugates of the compositions and methods comprise 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 monomers. In some embodiments the conjugates are between 2-8, 8-10, 10-15, 15-20, 18-20, 18-25, 15-25, 10-30, 10-35, 20-25, 25-30, 30-35, or less than 50 monomers. In still further embodiments, the conjugates are about 5 monomers, about 6 monomers, about 7 monomers, about 8 monomers, about 9 monomers, about 10 monomers, about 11 monomers, about 12 monomers, about 13 monomers, about 14 monomers, about 15 monomers, about 16 monomers, about 17 monomers, about 18 monomers, about 19 monomers, about 20 monomers, about 21 monomers, about 22 monomers, about 23 monomers, about 24 monomers, about 25 monomers, about 26 monomers, about 27 monomers, about 28 monomers, about 29 monomers, about 30 monomers, about 31 monomers, about 32 monomers, about 33 monomers, about 34 monomers, about 35 monomers, about 36 monomers, about 37 monomers, about 38 monomers, about 39 monomers, or about 40 monomers. In some embodiments, the sequence-defined polymer backbone comprises 5 monomers. In some embodiments, the sequence-defined polymer backbone comprises 10 monomers. In some embodiments, the sequence-defined polymer backbone comprises 15 monomers. In some embodiments, the sequence-defined polymer backbone comprises 20 monomers. In some embodiments, the sequence-defined polymer backbone comprises 25 monomers. In some embodiments, the sequence-defined polymer backbone comprises 30 monomers. In some embodiments, the sequence-defined polymer backbone comprises 35 monomers. In some embodiments, the number of monomers comprising the sequence-defined polymer backbone is even. In some embodiments, the number of monomers comprising the sequence-defined polymer backbone is odd. In some embodiments, the sequence-defined polymer backbone comprises 35 serinol-phosphoramidite monomers.

In some embodiments of the disclosure, the compositions and methods provides for conjugates comprising a sequence-defined polymer backbone having 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 side chains per backbone.

In some embodiments, the monomers comprising the sequence-defined polymer backbone has a defined number of possible side chain conjugation sites. In some embodiments, the number of conjugation sites per sequence-defined polymer is at least about 5, at least about 10, at least about 15, at least about 20, at least about 25, at least about 30, at least about 35, at least about 40, at least about 45, at least about 50, or at least about 55. In some embodiments, each monomer comprising the sequence-defined polymer backbone is covalently linked to at least about 5 side chains, at least about 6 side chains, at least about 7 side chains, at least about 8 side chains, at least about 9 side chains, at least about 10 side chains, at least about 11 side chains, at least about 12 side chains, at least about 13 side chains, at least about 14 side chains, at least about 15 side chains, at least about 16 side chains, at least about 17 side chains, at least about 18 side chains, at least about 19 side chains, at least about 20 side chains, at least about 21 side chains, at least about 22 side chains, at least about 23 side chains, at least about 24 side chains, at least about 25 side chains, at least about 26 side chains, at least about 27 side chains, at least about 28 side chains, at least about 29 side chains, at least about 30 side chains, at least about 31 side chains, at least about 32 side chains, at least about 33 side chains, at least about 34 side chains, at least about 35 side chains, at least about 36 side chains, at least about 37 side chains, at least about 38 side chains, at least about 39 side chains, or at least about 40 side chains. In some embodiments, each monomer comprising the sequence-defined polymer backbone is covalently linked to about 5-35 side chains.

In some embodiments, the percentage of side chains covalently linked to the possible conjugation sites available on the sequence-defined polymer backbone is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100%.

In some embodiments, the compositions and methods provide for a conjugate comprising at least one side chain that is a polysaccharide, a zwitterion polymer, or polyethylene glycol (PEG). In some embodiments, the polysaccharide side chain is amylose. In some embodiments, the polysaccharide side chain is hyaluronic acid. In some embodiments, the zwitterion polymer is poly(methacryloyl-L-lysine, poly(sulfobetaine methacrylate), or poly(carboxybetaine methacrylate).

In some embodiments, the compositions and methods provide for at least one oligonucleotide linked to a terminal end of the sequence-defined polymer (i.e., linking the at least one oligonucleotide to a monomer wherein the monomer is linked to only one monomer). As used herein, such a conjugate formation may be referred to as a “bottlebrush,” “pacDNA,” or “conjugate.”

In some embodiments, the compositions and method provide for at least one oligonucleotide linked to the terminal end of a first sequence-defined polymer and linked the terminal end of a second sequence-defined polymer. As used herein, such a conjugate formation may be referred to as a “dumb-brush,” a “dumbbell-like brush,” or a “dumbbell-like pacDNA.”

In some embodiments, the compositions and methods provide for at least one oligonucleotide linked to a non-terminal end of a sequence-defined polymer (i.e., linking the at least one oligonucleotide to a monomer wherein the monomer is linked to at least two other monomers). As used herein, this conjugate formation is referred to as a “doubler,” a “doubler-brush,” or a “doubler pacDNA.”

In some embodiments, the compositions and methods provide for at least one oligonucleotide linked to a non-terminal site of the backbone, e.g., the middle of the backbone.

In some embodiments, the compositions and methods provide for an oligonucleotide that is sufficiently complementary to a target polynucleotide. As used herein, the term “complementary” means that one oligonucleotide is identical to, or hybridizes selectively to, another oligonucleotide. In one alternative embodiment, one oligonucleotide hybridizes specifically to the other oligonucleotide. See M. Kanehisa, Nucleic Acids Res. 12:203 (1984). In some embodiments, the oligonucleotide is sufficiently complementary to a target polynucleotide to hybridize to the target polynucleotide. In some embodiments, the oligonucleotide can bind to non-nucleic acid target under predetermined conditions.

In some embodiments, the target sequence is associated with a disease or disorder. In some embodiments, the disease or disorder is a genetic disease or disorder associated with a genetic variant selected from a single-nucleotide polymorphism (SNP), substitution, insertion, deletion, transition, transversion, translocation, nonsense, missense, and/or frameshift mutation.

In some embodiments, the disease or disorder the disease or disorder is non-small cell lung carcinoma, ovarian carcinoma, advanced cancer, severe psoriasis, Duchenne muscular dystrophy, thromboembolism, progeria (Hutchinson-Gilford progeria syndrome (HGPS)), recessive dystrophic epidermolysis bullosa, Pompe's disease, a non-liver disorder, a multisystem disorder, or any combination thereof.

In some embodiments, the compositions and methods of the disclosure provide for at least one oligonucleotide that is an aptamer, a single-stranded DNA, a double-stranded DNA, a single-stranded RNA, a double stranded RNA, a ribozyme, a DNAzyme, an antisense oligonucleotide, an exon-skipping oligonucleotide, an siRNA oligonucleotide, a triple helix forming oligonucleotide, or a combination thereof. In some embodiments, the oligonucleotide is chemically modified, i.e., has a chemical structure that deviates from the natural DNA or RNA structure in the internucleotide linkage, a nucleobase, or a pentose, or a combination thereof.

In some embodiments, at least one oligonucleotide comprises deoxyribonucleotides. In certain embodiments, the oligonucleotide comprises ribonucleotides. Non-limiting examples of oligonucleotide include single-, double- or multi-stranded DNA or RNA, DNA-RNA hybrids, or a polymer comprising purine and pyrimidine bases, or other natural, i or biochemically modified, non-natural, or derivatized nucleotide bases. The backbone of the oligonucleotide can comprise sugars and phosphate groups, modified or substituted sugar or phosphate groups, a polymer of synthetic subunits such as phosphoramidates, or a combination thereof.

In some embodiments, at least one oligonucleotide is isolated (e.g., produced synthetically or via molecular cloning). In some embodiments, at least one oligonucleotide is integrated into the genomic DNA of a host cell (e.g., a T lymphocyte). In some embodiments, at least one oligonucleotide is extrachromosomal (e.g., on a plasmid, on a viral vector) within a host cell. In some embodiments, at least one oligonucleotide is a DNA. In some embodiments, at least one oligonucleotide is an RNA. In addition, at least one oligonucleotide can include one or more modified nucleotides (e.g., one or more chemically modified nucleotides). In some embodiments, the oligonucleotide is between 8-20, between 8-10, between 10-20, between 10-30, between 10-40, between 15-20, between 15-30, or between 20-30 nucleotides long. In some embodiments, at least one oligonucleotide further comprises at least one detectable label. In some embodiments, the detectable label is a naturally occurring nucleic acid, a synthetic nucleic acid, a chemically modified nucleic acid, a fluorescent dye, or a radioactive dye. In some embodiments, the detectable label is Cy5.

In some embodiments, at least one oligonucleotide is linked to the monomer and/or sequence-defined polymer backbone via a cleavable bond. In some embodiments, the conjugate comprises two or more oligonucleotides. In some embodiments, the conjugate comprises three, four, five, six, seven, or more oligonucleotides. In some embodiments, the oligonucleotides are identical, substantially identical, or substantially distinct.

In some embodiments, at least one oligonucleotide comprises a linker used for conjugation to the polymer backbone and is otherwise unmodified (e.g., natural DNA or RNA).

As used herein, the term “sequence identity,” refers to the extent to which two sequences have the same residues at the same positions when the sequences are aligned to achieve a maximal level of identity, expressed as a percentage. For sequence alignment and comparison, typically one sequence is designated as a reference sequence, to which a test sequences are compared. Sequence identity between reference and test sequences is expressed as a percentage of positions across the entire length of the reference sequence where the reference and test sequences share the same nucleotide or amino acid upon alignment of the reference and test sequences to achieve a maximal level of identity. As an example, two sequences are considered to have 70% sequence identity when, upon alignment to achieve a maximal level of identity, the test sequence has the same nucleotide residue at 70% of the same positions over the entire length of the reference sequence.

Alignment of sequences for comparison to achieve maximal levels of identity can be readily performed by a person of ordinary skill in the art using an appropriate alignment method or algorithm. In some instances, alignment can include introduced gaps to provide for the maximal level of identity. Examples include the local homology algorithm of Smith & Waterman, Adv. Appl. Math. 2:482 (1981), the homology alignment algorithm of Needleman & Wunsch, J. Mol. Biol. 48:443 (1970), the search for similarity method of Pearson & Lipman, Proc. Natl. Acad. Sci. USA 85:2444 (1988), computerized implementations of these algorithms (GAP, BESTFIT, FASTA, and TFASTA in the Wisconsin Genetics Software Package, Genetics Computer Group, 575 Science Dr., Madison, Wis.), and visual inspection (see generally Ausubel et al., Current Protocols in Molecular Biology). In some embodiments, codon-optimized sequences for efficient expression in different cells, tissues, and/or organisms reflect the pattern of codon usage in such cells, tissues, and/or organisms containing conservative (or non-conservative) amino acid substitutions that do not adversely affect normal activity.

In some embodiments, the conjugate comprises a backbone that is a poly(serinol phosphodiester) (PSP) backbone comprising at least two serinol-phosphoramidites. In some embodiments, the backbone is a poly(serinol phosphodiester) (PSP) backbone comprising 5, 10, 15, 20, 25, 30, 35, 40, 45, or 50 serinol-phosphoramidites. In some embodiments, the backbone is a poly(serinol phosphodiester) (PSP) backbone comprising 30 serinol-phosphoramidites.

In some embodiments, the compositions and methods further comprise a pharmaceutically acceptable carrier. As used herein, the term “pharmaceutically acceptable” refers to species which are, within the scope of sound medical judgment, suitable for use without undue toxicity, irritation, allergic response and the like, and are commensurate with a reasonable benefit/risk ratio. For example, a substance is pharmaceutically acceptable when it is suitable for use in contact with cells, tissues or organs of animals or humans without excessive toxicity, irritation, allergic response, immunogenicity or other adverse reactions, in the amount used in the dosage form according to the dosing schedule, and commensurate with a reasonable benefit/risk ratio.

In some embodiments of the disclosure, the compositions and methods provide for a method of treating a disease or disorder, comprising administering to a subject in need thereof, a therapeutically effective amount of a bottlebrush polymer-oligonucleotide conjugate (conjugate).

As used herein, “therapy,” “treat,” “treating,” or “treatment” means inhibiting or relieving a condition in a subject in need thereof. For example, a therapy or treatment refers to any of: (i) the prevention of symptoms associated with a disease or disorder (e.g., cancer); (ii) the postponement of development of the symptoms associated with a disease or disorder (e.g., cancer); and/or (iii) the reduction in the severity of such symptoms that will, or are expected, to develop with said disease or disorder (e.g., cancer). The terms include ameliorating or managing existing symptoms, preventing additional symptoms, and ameliorating or preventing the underlying causes of such symptoms. Thus, the terms denote that a beneficial result is being conferred on at least some of the subjects (e.g., humans) being treated. Many therapies or treatments are effective for some, but not all, subjects that undergo the therapy or treatment.

As used herein, the term “effective amount” means an amount of a composition, that when administered alone or in combination to a cell, tissue, or subject, is effective to achieve the desired therapy or treatment under the conditions of administration. For example, an effective amount is one that would be sufficient to produce an immune response to bring about effectiveness of a therapy (therapeutically effective) or treatment. The effectiveness of a therapy or treatment (e.g., eliciting a humoral and/or cellular immune response) can be determined by suitable methods known in the art.

In some embodiments, compositions as described herein are used in combination with other known agents (e.g., additional therapeutic agents) and therapies, such as chemotherapy, transplantation, and radiotherapy. Administered “in combination”, as used herein, means that two (or more) different treatments are delivered to the subject during the course of the subject's treatment e.g., the two or more treatments are delivered after the subject has been diagnosed with the disease and before the disease has been cured or eliminated or treatment has ceased for other reasons. In some embodiments, different treatments (e.g., additional therapeutics) can be administered simultaneously or sequentially.

As used herein, “subject” or “patient” includes humans, domestic animals, such as laboratory animals (e.g., dogs, monkeys, pigs, rats, mice, etc.), household pets (e.g., cats, dogs, rabbits, etc.) and livestock (e.g., chickens, pigs, cattle (e.g., a cow, bull, steer, or heifer), sheep, goats, horses, etc.), and non-domestic animals. In some embodiments, a subject is a mammal (e.g., a non-human mammal). In some embodiments, a subject is a human. In still further embodiments, a subject of the disclosure may be a cell, cell culture, tissue, organ, or organ system.

In some embodiments the subject is about 0-3 months, 0-6 months, 6-11 months, 12-15 months, 12-18 months, 19-23 months, 24 months, 1-2 years, 2-3 years, 4-6 years, 7-10 years, 11-12 years, 11-15 years, 16-18 years, 18-20 years, 20-25 years, 25-30 years, 30-35 years, 30-40 years, 35-40 years, 30-50 years, 30-60 years, 50-60 years, 60-70 years, 50-80 years, 70-80 years, 80-90 years, or older than 60 years.

In still further embodiments, the method comprises administering to the subject an effective amount of the composition, or a pharmaceutically acceptable salt thereof.

The term “pharmaceutically acceptable salts” embraces salts commonly used to form alkali metal salts and to form addition salts of free acids or free bases. The nature of the salt is not critical, provided that it is pharmaceutically acceptable.

Suitable pharmaceutically acceptable acid addition salts may be prepared from an inorganic acid or an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, arylaliphatic, heterocyclic, carboxylic and sulfonic classes of organic acids, examples of which are formic, acetic, propionic, succinic, glycolic, gluconic, maleic, embonic (pamoic), methanesulfonic, ethanesulfonic, 2-hydroxyethanesulfonic, pantothenic, benzenesulfonic, toluenesulfonic, sulfanilic, mesylic, cyclohexylaminosulfonic, stearic, algenic, β-hydroxybutyric, malonic, galactic, and galacturonic acid. Pharmaceutically acceptable acidic/anionic salts also include, the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, malonate, mandelate, mesylate, methylsulfate, mucate, napsylate, nitrate, pamoate, pantothenate, phosphate/diphospate, polygalacturonate, salicylate, stearate, subacetate, succinate, sulfate, hydrogensulfate, tannate, tartrate, teoclate, tosylate, and triethiodide salts.

Suitable pharmaceutically acceptable base addition salts include, but are not limited to, metallic salts made from aluminum, calcium, lithium, magnesium, potassium, sodium and zinc or organic salts made from N,N′-dibenzylethylene-diamine, chloroprocaine, choline, diethanolamine, ethylenediamine, N-methylglucamine, lysine, arginine and procaine. Pharmaceutically acceptable basic/cationic salts also include, the diethanolamine, ammonium, ethanolamine, piperazine and triethanolamine salts.

All of these salts may be prepared by conventional means by treating, for example, a composition described herein with an appropriate acid or base.

A “pharmaceutical composition” refers to a formulation of one or more therapeutic agents and a medium generally accepted in the art for delivery of a biologically active agent to subjects, e.g., humans. In some embodiments, a pharmaceutical composition may include one or more pharmaceutically acceptable excipients, diluents, or carriers. “Pharmaceutically acceptable carrier, diluent, or excipient” includes any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in subjects.

In some embodiments, the pharmaceutical composition is formulated as a solution.

“Pharmaceutically acceptable carrier” refers to an ingredient in a pharmaceutical composition, other than an active ingredient, which is nontoxic to a subject. A pharmaceutically acceptable carrier includes, but is not limited to, a buffer, excipient, stabilizer, or preservative. In some embodiments, the carrier may be a diluent, adjuvant, excipient, or vehicle with which the agent (e.g., oligonucleotide) is administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine can be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the agent in such pharmaceutical formulation may vary widely, i.e., from less than about 0.5%, to at least about 1%, or to as much as 15% or 20%, 25%, 30%, 35%, 40%, 45% or 50% by weight. The concentration will be selected primarily based on required dose, fluid volumes, viscosities, etc., according to the mode of administration. Suitable vehicles and formulations, inclusive of other human proteins, e.g., human serum albumin, are described, for example, in Remington: The Science and Practice of Pharmacy, 21^{st} Edition, Troy, D. B. ed., Lipincott Williams and Wilkins, Philadelphia, PA 2006, Part 5, Pharmaceutical Manufacturing: 691-1092 (e.g., pages 958-89).

In some embodiments, a pharmaceutical composition suitable for use in methods of the disclosure further comprises one or more pharmaceutically acceptable carriers. The term “pharmaceutically acceptable carrier” refers to an ingredient in a pharmaceutical composition, other than an active ingredient, which is nontoxic to a subject and should not interfere with the efficacy of the active ingredient. A pharmaceutically acceptable carrier includes, but is not limited to, such as those widely employed in the art of drug manufacturing. The carrier may be a diluent, adjuvant, excipient, or vehicle with which the agent is administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine may be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the agent in such pharmaceutical formulation may vary widely, e.g., from less than about 0.5%, usually to at least about 1% to as much as 15%, 20%, 25%, 30%, 35%, 40%, 45% or 50% by weight. The concentration will be selected primarily based on required dose, fluid volumes, viscosities, etc., according to the particular mode of administration selected. Suitable vehicles and formulations, inclusive of other human proteins, e.g., human serum albumin, are described, for example, in e.g., Remington: The Science and Practice of Pharmacy, 21^{st} Edition, Troy, D. B. ed., Lipincott Williams and Wilkins, Philadelphia, Pa. 2006, Part 5, Pharmaceutical Manufacturing pp 691-1092, see especially pp. 958-89.

Non-limiting examples of pharmaceutically acceptable carriers are solvents, dispersion media, coatings, antibacterial and antifungal agents, isotonic and absorption delaying agents, and the like that are physiologically compatible, such as salts, buffers, antioxidants, saccharides, aqueous or non-aqueous carriers, preservatives, wetting agents, surfactants or emulsifying agents, or combinations thereof.

Non-limiting examples of buffers that may be used are acetic acid, citric acid, formic acid, succinic acid, phosphoric acid, carbonic acid, malic acid, aspartic acid, histidine, boric acid, Tris buffers, HEPPSO and HEPES.

Non-limiting examples of antioxidants that may be used are ascorbic acid, methionine, cysteine hydrochloride, sodium bisulfate, sodium metabisulfite, sodium sulfite, lecithin, citric acid, ethylenediamine tetraacetic acid (EDTA), sorbitol and tartaric acid.

Non-limiting examples of amino acids that may be used are histidine, isoleucine, methionine, glycine, arginine, lysine, L-leucine, tri-leucine, alanine, glutamic acid, L-threonine, and 2-phenylamine.

Non-limiting examples of surfactants that may be used are polysorbates (e.g., polysorbate-20 or polysorbate-80); polyoxamers (e.g., poloxamer 188); Triton; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g., lauroamidopropyl); myristamidopropyl-, palmidopropyl-, or isostearamidopropyl-dimethylamine; sodium methyl cocoyl-, or disodium methyl oleyl-taurate; and the MONAQUA™ series (Mona Industries, Inc., Paterson, N.J.), polyethyl glycol, polypropyl glycol, and copolymers of ethylene and propylene glycol (e.g., PLURONICS™, PF68, etc.).

Non-limiting examples of preservatives that may be used are phenol, m-cresol, p-cresol, o-cresol, chlorocresol, benzyl alcohol, phenylmercuric nitrite, phenoxyethanol, formaldehyde, chlorobutanol, magnesium chloride, alkylparaben (methyl, ethyl, propyl, butyl and the like), benzalkonium chloride, benzethonium chloride, sodium dehydroacetate and thimerosal, or mixtures thereof.

Non-limiting examples of saccharides that may be used are monosaccharides, disaccharides, trisaccharides, polysaccharides, sugar alcohols, reducing sugars, nonreducing sugars such as glucose, sucrose, trehalose, lactose, fructose, maltose, dextran, glycerin, dextran, erythritol, glycerol, arabitol, sylitol, sorbitol, mannitol, mellibiose, melezitose, raffinose, mannotriose, stachyose, maltose, lactulose, maltulose, glucitol, maltitol, lactitol or iso-maltulose.

Non-limiting examples of salts that may be used are acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like. In some embodiments, the salt is sodium chloride (NaCl).

Agents (e.g., oligonucleotide) disclosed herein may be prepared in accordance with standard procedures and are administered at dosages that are selected to reduce, prevent, or eliminate, or to slow or halt progression of, a condition being treated (See, e.g., Remington's Pharmaceutical Sciences, Mack Publishing Company, Easton, PA, and Goodman and Gilman's The Pharmaceutical Basis of Therapeutics, McGraw-Hill, New York, N.Y., the contents of which are incorporated herein by reference, for a general description of methods for administering various agents for human therapy).

In some embodiments, compositions of the disclosure are administered in a delivery vehicle comprising a nanocarrier selected from the group consisting of a lipid, a polymer and a lipo-polymeric hybrid. In still further embodiments, the first and second polynucleotides are encapsulated in a lipid nanoparticle, polymer nanoparticle, virus-like particle, nanowire, exosome, or hybrid lipid/polymer nanoparticle. In some embodiments, the first and second polynucleotides are encapsulated in the same nanocarrier. In still further embodiments, the first and second polynucleotides are encapsulated in different nanocarriers. In some embodiments, the lipid nanoparticle is ionizable.

As used herein, the term “pharmaceutically acceptable” refers to species which are, within the scope of sound medical judgment, suitable for use without undue toxicity, irritation, allergic response and the like, and are commensurate with a reasonable benefit/risk ratio. For example, a substance is pharmaceutically acceptable when it is suitable for use in contact with cells, tissues or organs of animals or humans without excessive toxicity, irritation, allergic response, immunogenicity or other adverse reactions, in the amount used in the dosage form according to the dosing schedule, and commensurate with a reasonable benefit/risk ratio.

A desired dose may conveniently be administered in a single dose, for example, such that the agent is administered once per day, or as multiple doses administered at appropriate intervals, for example, such that the agent is administered 2, 3, 4, 5, 6 or more times per day. The daily dose can be divided, especially when relatively large amounts are administered, or as deemed appropriate, into several, for example 2, 3, 4, 5, 6 or more, administrations. Typically, the compositions will be administered from about 1 to about 6 (e.g., 1, 2, 3, 4, 5 or 6) times per day or, alternatively, as an infusion (e.g., a continuous infusion). In some embodiments, the administration of the conjugate may be repeated after one day, two days, three days, four days, five days, six days, one week, two weeks, three weeks, four weeks, five weeks, six weeks, seven weeks, two months, three months, four months, five months, six months or longer. The repeated administration may be at the same dose or at a different dose.

Determining the dosage and route of administration for a particular agent, patient and disease or condition is well within the abilities of one of skill in the art. In certain embodiments, the administration of the composition may be carried out in any manner, e.g., by parenteral or nonparenteral administration, including by aerosol inhalation, injection, infusions, ingestion, transfusion, implantation or transplantation. For example, the compositions described herein may be administered to a patient trans-arterially, intradermally, subcutaneously, intratumorally, intramedullary, intranodally, intramuscularly, by intravenous (i.v.) injection, intranasally, intrathecally or intraperitoneally. In one aspect, the compositions of the present disclosure are administered intravenously. In one aspect, the compositions of the present disclosure are administered to a subject by intramuscular or subcutaneous injection. The compositions may be injected, for instance, directly into a tumor, lymph node, tissue, organ, or site of infection. In some embodiments the route of administration is intramuscular, intranodal, intravenous, intradermal, subcutaneous, intranasal, infusion, intraperitoneal, intracranial, intratracheal or epicardial. Preferably, the dosage does not cause or produces minimal adverse side effects.

In some embodiments the route of administration is determined by the tissue or tissues, or organ to which the agent or agents are targeted. In some embodiments, the tissue, tissues, or organ is the subject's lung, ovary, immune system, skin, blood vessel, muscle, blood, brain, heart, intestine(s), pancreas, spleen, kidney, heart, bone, bone marrow, stomach, head, or any combination thereof.

Doses lower or higher than those recited above may be required. Specific dosage and treatment regimens for any particular subject will depend upon a variety of factors, for example, the activity of the specific agent employed, the age, body weight, general health status, sex, diet, time of administration, rate of excretion, drug combination, the severity and course of the disease, condition or symptoms, the subject's disposition to the disease, condition or symptoms, the judgment of the treating physician and the severity of the particular disease being treated. The amount of an agent in a composition will also depend upon the particular agent in the composition.

In some embodiments, the disclosure provides for compositions and methods of modulating or altering the expression of a gene product encoded by a target polynucleotide. viral polynucleotides. In some embodiments, the target polynucleotide is a polynucleotide specific to a mammalian cancer cell, a mammalian non-cancer cell, a plant cell, a bacterium, or a virus. In some embodiments, administration to the subject occurs in the absence of a transfection vector. In some embodiments, efficacy of administration is determined by measuring the subject's plasma pharmacokinetics, blood availability, extrahepatic distribution, tissue retention, dosing frequency or amount, or a combination thereof.

In some embodiments, the disclosure provides for a library of bottlebrush polymer-oligonucleotides conjugates and/or sequence-defined polymer backbones. In still further embodiments, the disclosure provides for a kit comprising at least one oligonucleotide and a sequence-defined polymer backbone.

Also included herein are methods of analyzing the physiochemical or biological properties (e.g., one or more of the properties described herein) of libraries of the conjugates described herein, and making predictions regarding backbone sequences which will impart desired or improved properties.

### EXEMPLIFICATION

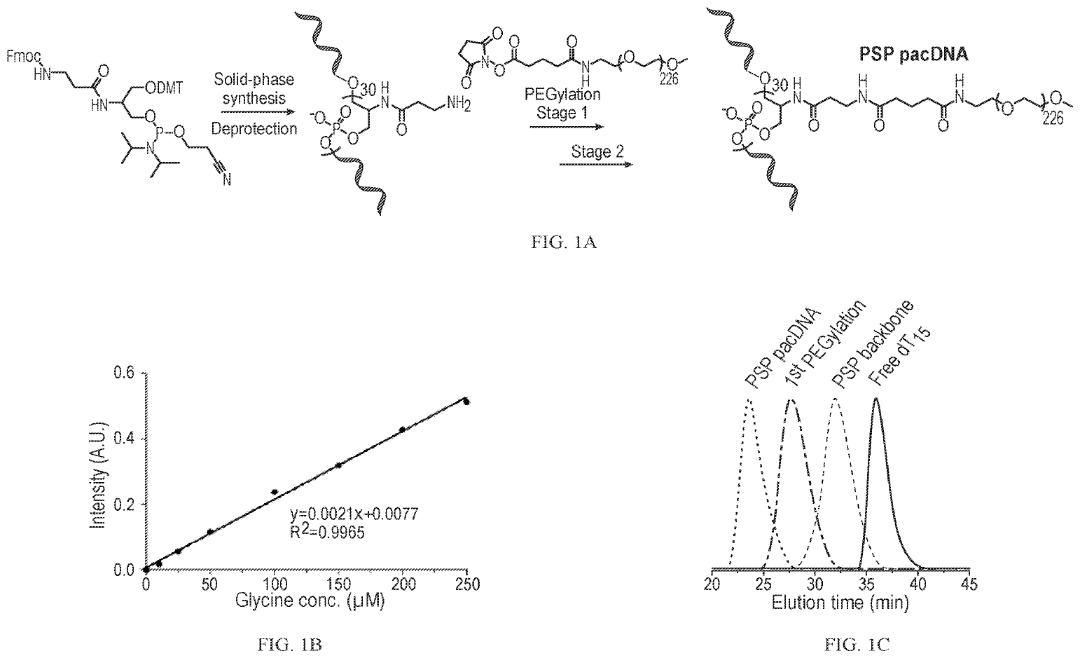

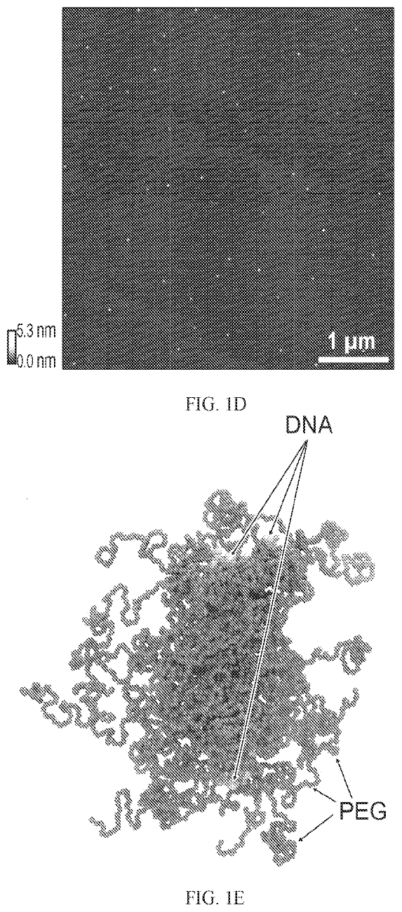

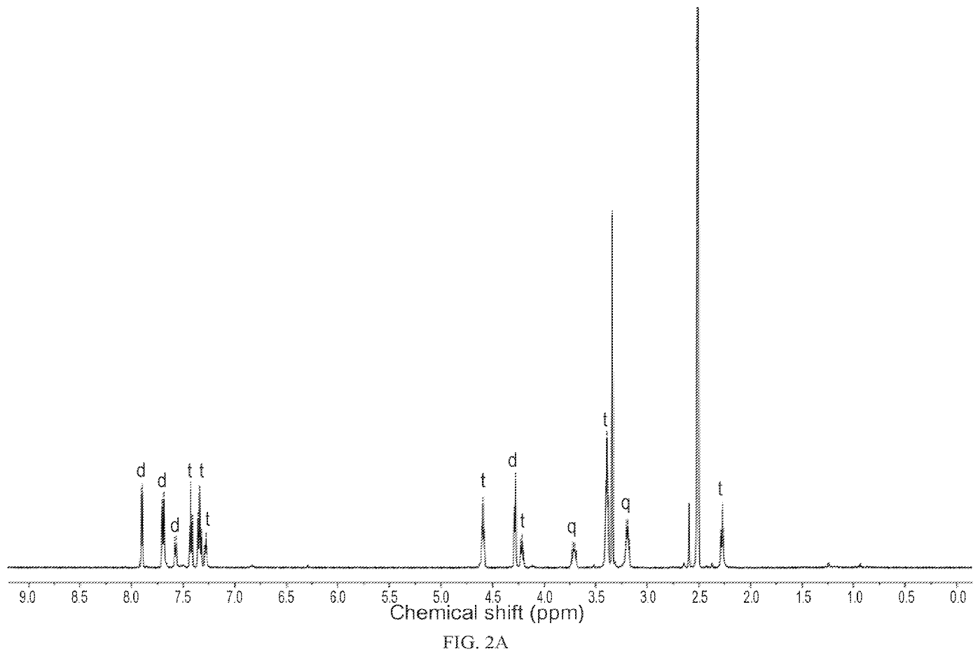

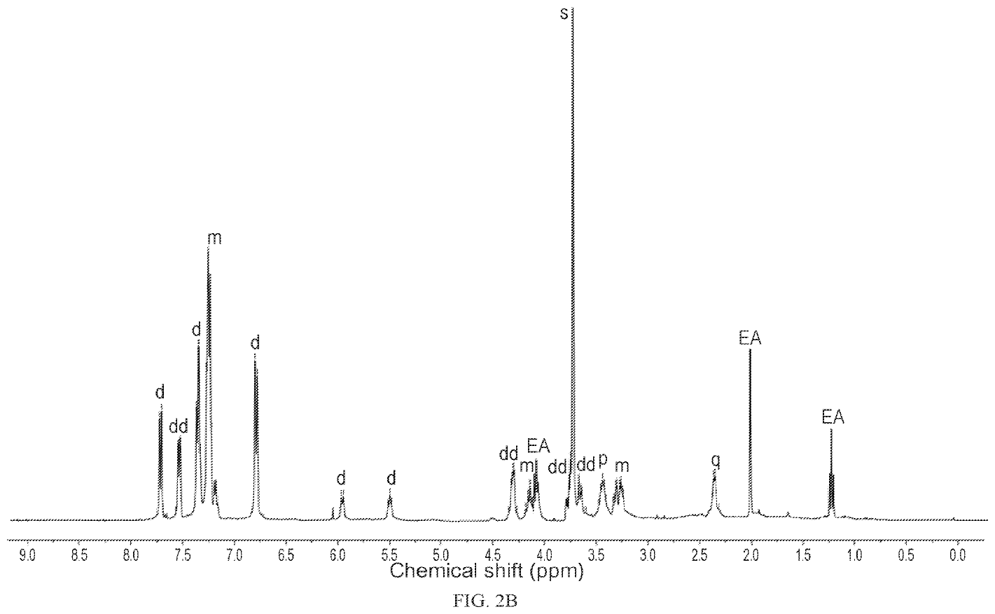

In an embodiment, polyphosphodiester (PPDE) backbone of the bottlebrush polymer is assembled by stepwise condensation of an Fmoc-protected phosphoramidite derived from serinol (FIG. 1A), a common intermediate for pharmaceuticals. Synthesis of the phosphoramidite building blocks were confirmed by matrix-assisted laser desorption ionization-time of flight mass spectrometry (MALDI-TOF MS) and ¹H-NMR (FIGS. 2A, 2B, 2C).

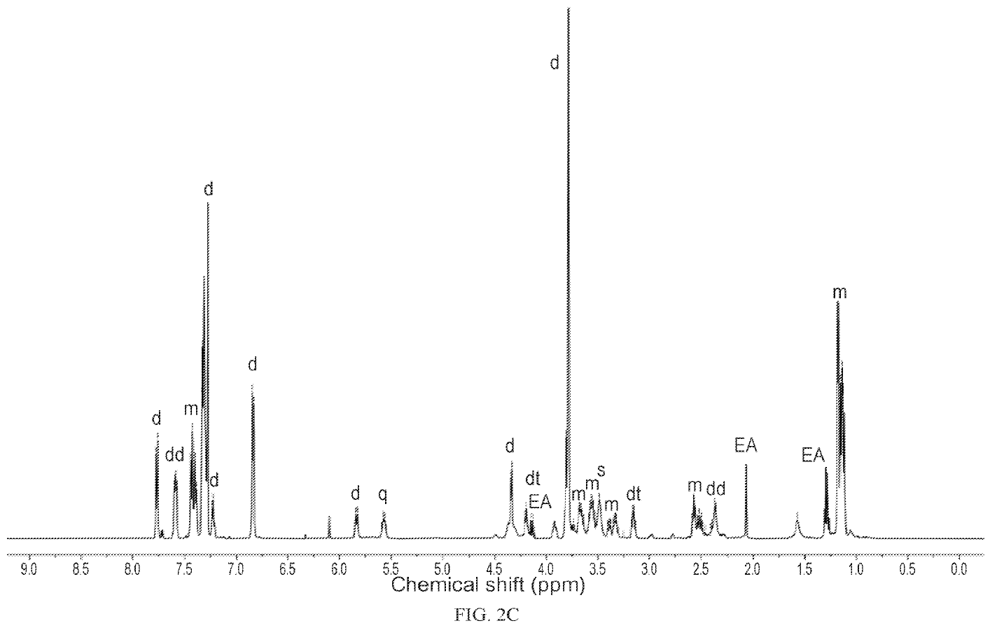

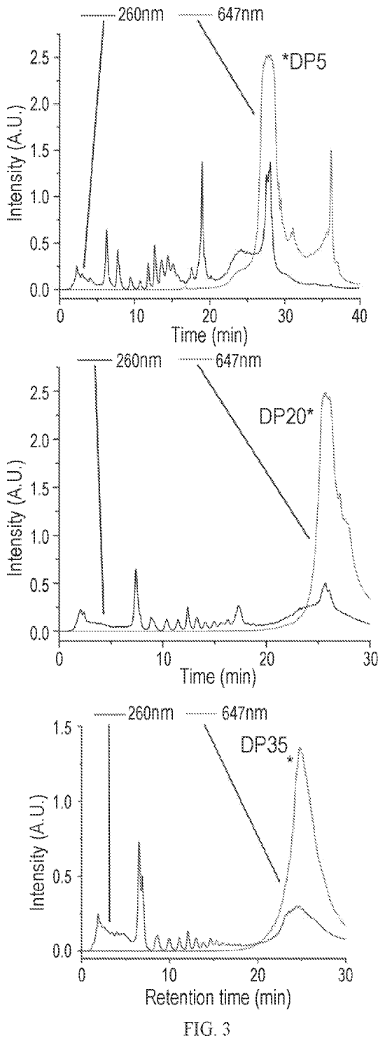

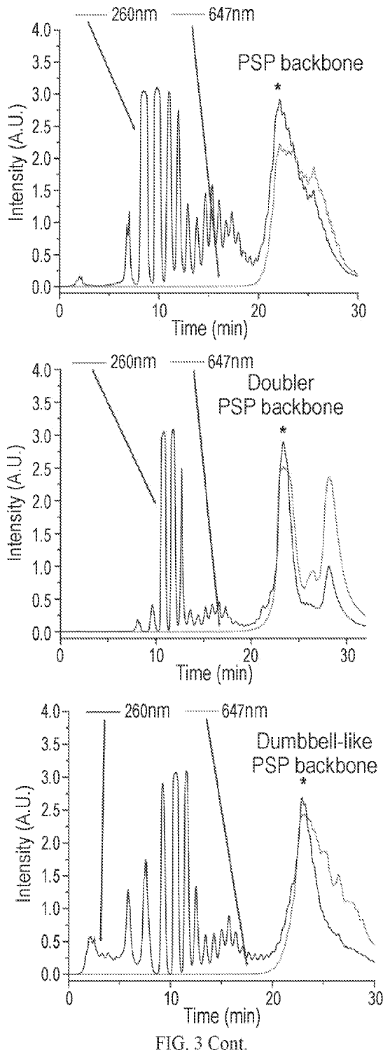

Because the synthesis of the polymer backbone shares the same chemistry as oligonucleotide synthesis, the aptamer portion can be prepared as part of the polymer backbone, eliminating subsequent aptamer conjugation and purification steps. For proof of concept, a poly(serinol phosphodiester) (PSP) backbone of 30 repeating units with two dT₁₅ strands flanking each terminus of the backbone was synthesized (dT₁₅-b-PSP₃₀-b-dT₁₅). Note that the serinol units are racemic. Following the synthesis, the Fmoc groups protecting the serinol amines were removed, and the strand was purified by reversed-phase high performance liquid chromatography (RP-HPLC; FIG. 3).

To construct the bottlebrush segment, the amine groups were derivatized with N-hydroxysuccinimide (NHS)-terminated PEG in a two-stage process. In the first stage, the purified backbone was treated with one equivalent of 10 kDa PEG succinimidyl glutaramide (1:1 amine:NHS ester) in 1× phosphate buffered saline (PBS, pH 7.4) at 4° C. overnight. Next, the product from the first stage was lyophilized and reacted with another equivalent of PEG (1:1 amine:NHS ester) in anhydrous N,N-dimethylformamide (DMF) for 60 h at room temperature. The two-stage PEGylation is necessary because NHS esters hydrolyze in an aqueous buffer leading to unsatisfactory coupling efficiencies, but without the initial aqueous coupling step, the anionic backbone/oligonucleotide sequence is insoluble in DMF. With the two-step process, near-quantitative coupling yields (≈95% conversion of serinol amines) were obtained as determined by a 2,4,6-trinitrobenzene sulfonic acid (TNBSA) assay using glycine as a standard (FIG. 1B and Table 1).

TABLE 1
The number of free amine groups on PSP backbones and the extent of
PEG derivatization after each stage as determined by TNBSA assay.

| | Amine # | Remaining | | Remaining | |
|---|---|---|---|---|---|
| | before | amine/polymer | Coupling | amine/polymer | Coupling |
| Sample ID | PEGylation | after 1^{st} Stage | efficacy | after 2^{nd} Stage | efficacy |
| DP5 | 5.3(±1.2) | 0.5(±0.2) | 90.9%(±4.8) | Not detected | 100% |
| DP20 | 19.5(±1.0) | 2.8(±0.7) | 86.0%(±3.6) | 1.9(±0.2) | 90.6%(±0.9) |
| DP35 | 32.3(±3.0) | 5.3(±0.6 | 85.0%(±1.7) | 2.0(±0.4) | 94.3%(±1.2) |
| PSP | 29.3(±1.8) | 4.6(±2.0) | 84.7%(±6.6) | 1.6(±0.2) | 94.7%(±0.7) |
| pacDNA | | | | | |
| Double PSP | 26.8(±1.2) | 3.6(±0.1) | 88.0%(±0.5) | 1.5(±0.6) | 94.9%(±2.0) |
| pacDNA | | | | | |
| Dumbbell- | 30.0(±4.0) | 5.4(±2.0) | 84.6%(±3.2) | 1.5(±0.1) | 95.2%(±0.4) |
| like PSP pac | | | | | |
| DNA | | | | | |

The two-stage PEGylation produced incremental increases in molecular weight (MW) after each coupling reaction, as observed by aqueous gel permeation chromatography (GPC) (FIG. 1C). Atomic Force Microscopy (AFM) measurement of the final structure, dT₁₅-b-(PSP₃₀-g-PEG)-b-dT₁₅ (termed PSP pacDNA) shows a spherical morphology with a drystate diameter of 21±3 nm (FIG. 1D and FIG. 1E).

The solid-phase methodology of PSP pacDNA synthesis carries significant advantages over the graft-through approach that have been adopted previously for PN pacDNA. For example, the degree of polymerization (DP) can be arbitrarily tuned by the number of synthesis cycles. Further, the synthesis provides access to additional molecular architectures, such as branched, dendritic, or block copolymers (or a combination thereof), which can be difficult to achieve with current polymer chemistry. Access to these new types of bottlebrush-DNA biohybrids, which can be very difficult with current polymer chemistry, will greatly expand the space for functional explorations that fulfill different goals.

To test the versatility of the approach, PSP bottlebrushes with different backbone DPs ranging from 5 to 35 (a 5′-Cy5 was used for quantification) were synthesized. See Table 2 and FIG. 1B for all PSP pacDNA structures and DNA sequences.

TABLE 2
Key PSP backbone sequences

| Sample ID | Sequence |
|---|---|
| DP5 | 5′-Cy5-XXX XXT-3′ |
| | (SEQ ID NO: 1) |
| DP20 | 5′-Cy5-XXX XXX XXX XXX XXX XXX XXT-3′ |
| | (SEQ ID NO: 2) |
| DP35 | 5′-Cy5-XXX XXX XXX XXX XXX XXX XXX XXX |
| | XXX XXX XXX XXT-3′ |
| | (SEQ ID NO: 3) |
| PSP pacDNA | 5′-Cy5-TTT TTT TTT TTT TTT XXX XXX XXX XXX |
| | XXX XXX XXX XXX XXX XXX TTT TTT TTT TTT |
| | TTT-3′ |
| | (SEQ ID NO: 4) |
| Double PSP pacDNA | (5′-XXX XXX XXX XXX XXX)2 D TTT TTT TTT TTT |
| | TTT-Cy5-3′ |
| | (SEQ ID NO: 5) |
| Dumbbell-like PSP | 5′-Cy5-XXX XXX XXX XXX XXX TTT TTT TTT TTT |
| pacDNA | TTT XXX XXX X XX XXX T-3′ |
| | (SEQ ID NO: 6) |
| dT15 | 5′-TTT TTT TTT TTT TTT-Cy5-3′ |
| | (SEQ ID NO: 7) |
| Quencher | 5′-Dabcyl-AAA AAA AAA AAA AAA-3′ |
| | (SEQ ID NO: 8) |
| Dummy quencher | 5′-Dabcyl-TTT TTT TTT TTT TTT-3′ |
| | (SEQ ID NO: 9) |
| HD1 | 5′-GGT TGG TGT GGT TGG-3′ |
| | (SEQ ID NO: 10) |
| HD1-DBCO | 5′-DBCO-TTT TTG GTT GGT GTG GTT GG-3′ |
| | (SEQ ID NO: 11) |
| PSP pacHD1 | 5′-GGT TGG TGT GGT TGG TTT TTX XXX XXX XXX |
| | XXX XXX XXX XXX XXX XXX XXT TTT |
| | TGG TTG GTG TGG TTG G -3′ |
| | (SEQ ID NO: 12) |
| PSP pacSCR | 5′-GGT GGT GGT TGT GGT TTT TTX XXX XXX XXX |
| | XXX XXX XXX XXX XXX XXX XXT TTT |
| | TGG TGG TGG TTG TGG -3′ |
| | (SEQ ID NO: 13) |
| Antidote | 5′-CCA ACC ACA CCA ACC-3′ |
| | (SEQ ID NO: 14) |
| Cy3-PSP pacDNA | 5′-Cy3-GCT ATT AGG AGT CTT TXX XXX XXX XXX |
| | XXX XXX XXX XXX XXX XXX XT-3′ |
| | (SEQ ID NO: 15) |
| Cy3-oligonucleotide- | 5′-DBCO-GCT ATT AGG AGT CTT T-Cy3-3′ |
| DBCO | (SEQ ID NO: 16) |
| Cy3-oligonucleotide | 5′-GCT ATT AGG AGT CTT T-Cy3-3′ |
| | (SEQ ID NO: 17) |
| 5′-Cy5-HD1 | 5′-Cy5-GGT TGG TGT GGT TGG-3′ |
| | (SEQ ID NO: 18) |
| 5′-Cy5-PSP pacHD1 | 5′-Cy5-GGT TGG TGT GGT TGG TTT TTX XXX XXX |
| | XXX XXX XXXX XXX XXX XXX XXT- |
| | 3′ |
| | (SEQ ID NO: 19) |
| 3′-Cy5-HD1 | 5′-GGT TGG TGT GGT TGG-Cy5-3′ |
| | (SEQ ID NO: 20) |
| 3′-Cy5-PSP pacHD1 | 5′-XXX XXX XXX XXX XXX XXX XXX XXX XXX |
| | XXX TTT TTG GTT GGT GTG GTT GG-Cy5-3′ |
| | (SEQ ID NO: 21) |

X: amine-serinol phosphoramidite; D: symmetric doubler phosphoramidite; DBCO: dibenzo-cyclooctyne phosphoramidite; underlined: LNA-modified bases.

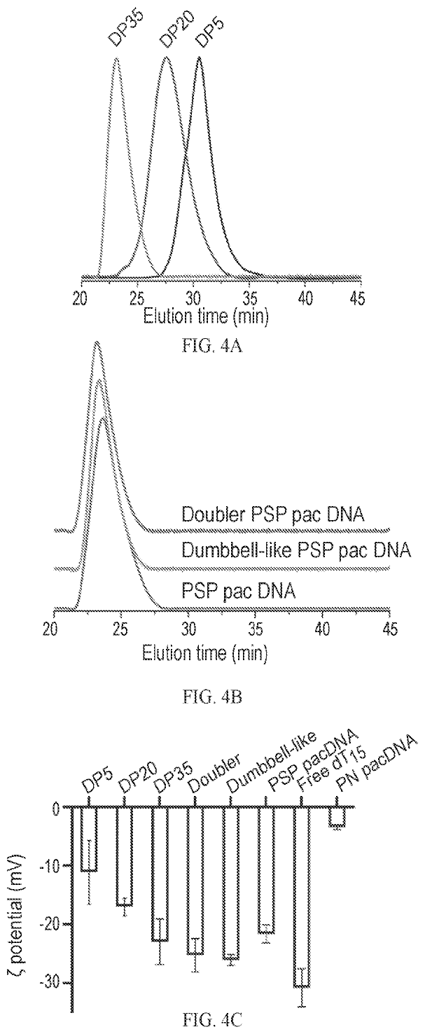

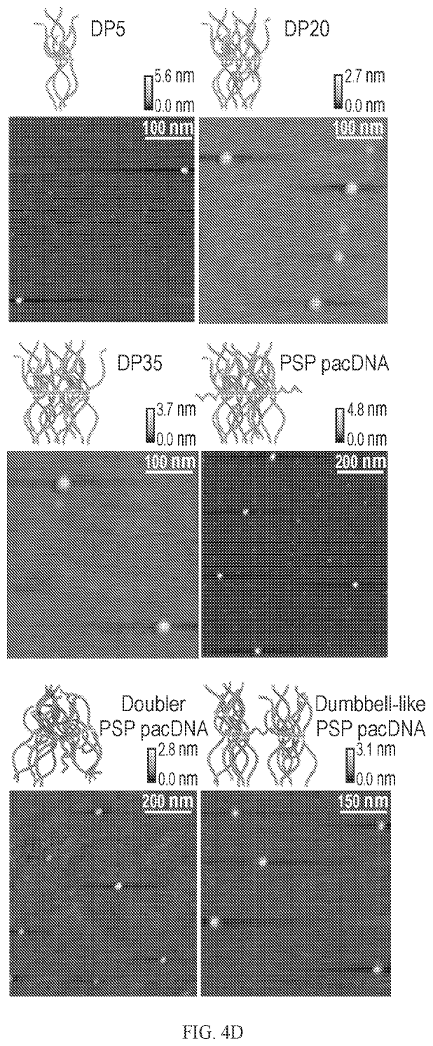

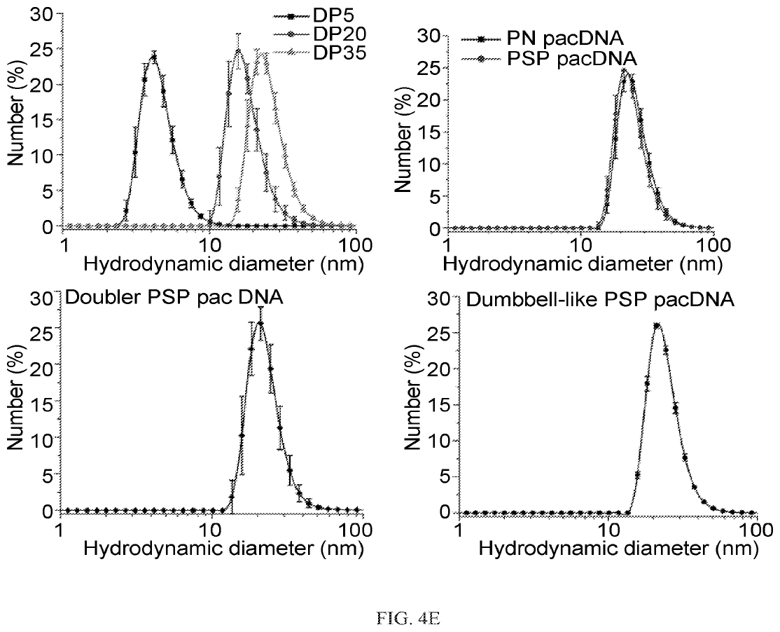

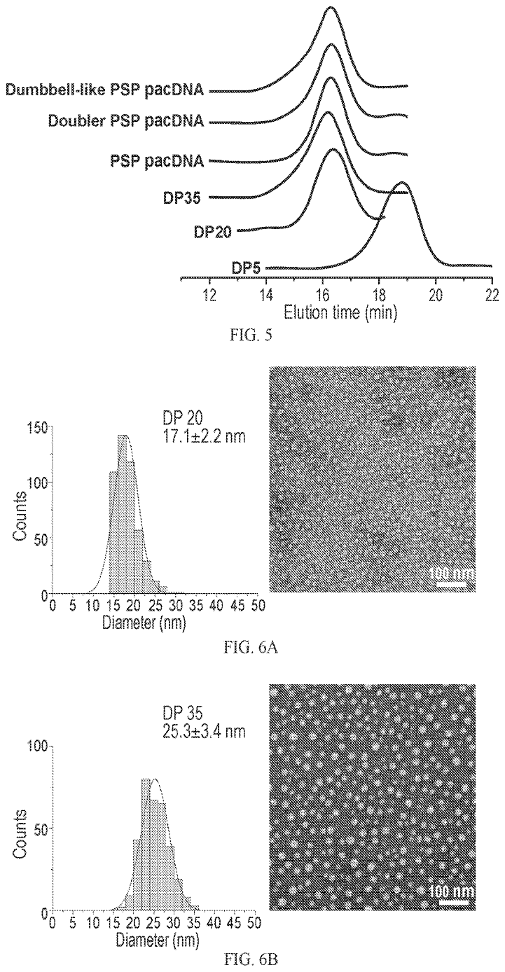

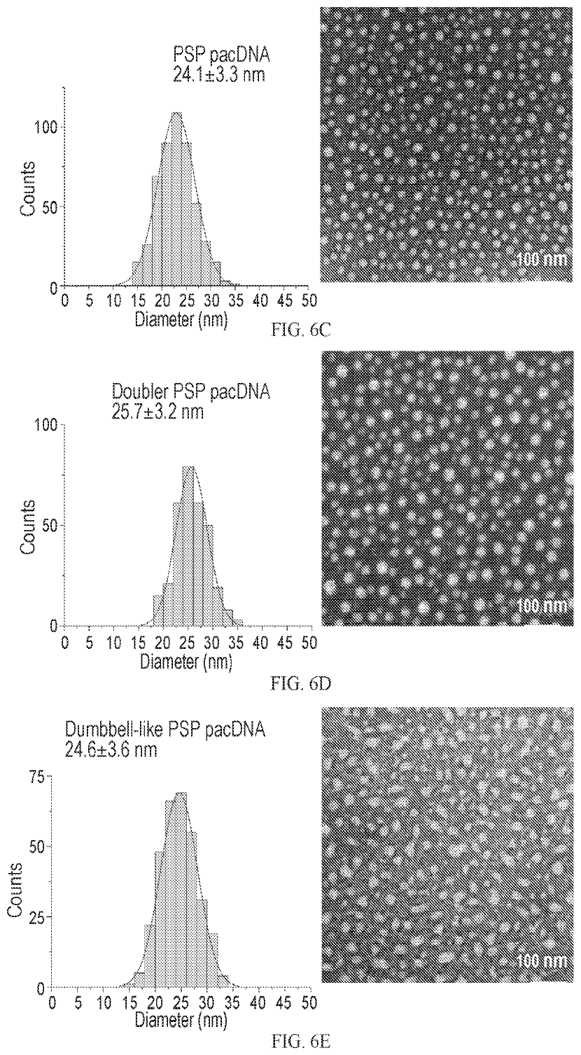

In addition, two architecturally distinct forms of PSP pacDNA were synthesized: (i) the dumbbell-like pacDNA, where the DNA strand is situated between two bottlebrush segments, and (ii) the doubler pacDNA, where the DNA is tethered via the 5′ to the middle unit of the brush backbone. The dumbbell-like pacDNA was synthesized with a dT₁₅ bridge between two PSP₁₅ segments. For the doubler pacDNA, a dT₁₅ segment was first synthesized normally (3′ to 5′), followed by the addition of a two-way branching unit (doubler phosphoramidite) at the 5′, upon which two serinol phosphoramidites were added in each coupling sequence. One benefit for the doubler pacDNA is that the brush backbone can be synthesized with half of the number of synthesis cycles while achieving the same total number of repeating units. TNBSA assay shows that the number of available amine groups associated with each backbone matches the expected value (Table 1). After the first stage of PEGylation, ≈85% of all backbone amine groups were consumed, and the yield increased to 90-100% after the second stage (Table 1). PSP-backboned bottlebrushes of DP 5, 20, and 35 show an increase in MW and size as evidenced by aqueous GPC (FIG. 4A) and dynamic light scattering (DLS) (FIG. 4E) measurements. Despite the different architectures and insertion positions of dT₁₅, PSP pacDNA and the structural variants of similar MW exhibit similar retention times in aqueous GPC (FIG. 4B) and hydrodynamic sizes (FIG. 4E). Narrow polydispersity indices (PDIs) in the range of 1.01-1.11 were observed for all samples. DMF GPC shows slightly higher PDIs in the range of 1.1-1.4 and larger-than-calculated MW, likely due to the polar bottlebrush backbone causing some aggregation in DMF (FIG. 5), manifesting in high MW tailing (Table 3). Both transmission electron microscopy (TEM, FIGS. 6A, 6B, 6C, 6D, 6E) and AFM (FIG. 4D and Table 4) confirm that these molecular brushes are non-aggregating and highly uniform in size in water, and particle size increases with backbone DPs.

TABLE 3
Average molecular weight (MW) of
PSP bottlebrushes and PSP pacDNAs

| Sample ID | Mₙ (kDa) | M_{w} (kDa) | PDI |
|---|---|---|---|
| DP5 | 48.9 | 68.1 | 1.39 |
| DP20 | 347.9 | 414.9 | 1.19 |
| DP35 | 421.8 | 582.9 | 1.38 |
| PSP pacDNA | 374.8 | 448.0 | 1.19 |
| Doubler PSP pacDNA | 374.6 | 442.5 | 1.18 |
| Dumbbell-like PSP pacDNA | 410.8 | 547.9 | 1.33 |

TABLE 4
Average dry-state size measurements
of PSP bottlebrushes and PSP pacDNAs

| Sample ID | Diameter (nm) |
|---|---|
| DP5 | 9.7(±1.8) |
| DP20 | 16.5(±1.5) |
| DP35 | 20(±2.4) |
| PSP pacDNA | 21.3(±3.0) |
| Doubler PSP pacDNA | 25.5(±2.9) |
| Dumbbell-like PSP pacDNA | 20.4(±2.9) |

Consisting of a multitude of phosphodiesters (pKa≈2.2), PSP bottlebrushes and PSP pacDNAs exhibit negative ζ potentials ranging from −11.1 to −26.1 mV under pH neutral conditions (Nanopure™ water) (FIG. 4C). Interestingly, PSP pacDNA and a PN-based counterpart of similar MW and hydrodynamic size (FIG. 4E and Table 5) exhibit very different ζ potentials: while the PSP materials show highly negative ζ potentials similar to free oligonucleotides, the PN-based material has a near-neutral ζ potential. Collectively, these results indicate that the PSP pacDNA synthesis is robust and can be used to prepare molecular brushes containing functional oligonucleotides with a high degree of structural freedom.

TABLE 5
Hydrodynamic diameter measurements of
PSP bottlebrushes and PSP pacDNAs

| Sample ID | Diameter (nm) |
|---|---|
| DP5 | 4.6(±0.2) |
| DP20 | 18.1(±1.4) |
| DP35 | 25.7(±1.0) |
| PSP pacDNA | 24.6(±1.0) |
| Doubler PSP pacDNA | 22.8(±1.5) |
| Dumbbell-like PSP pacDNA | 24.3(±0.1) |
| PN pacDNA | 25.7(±0.9) |

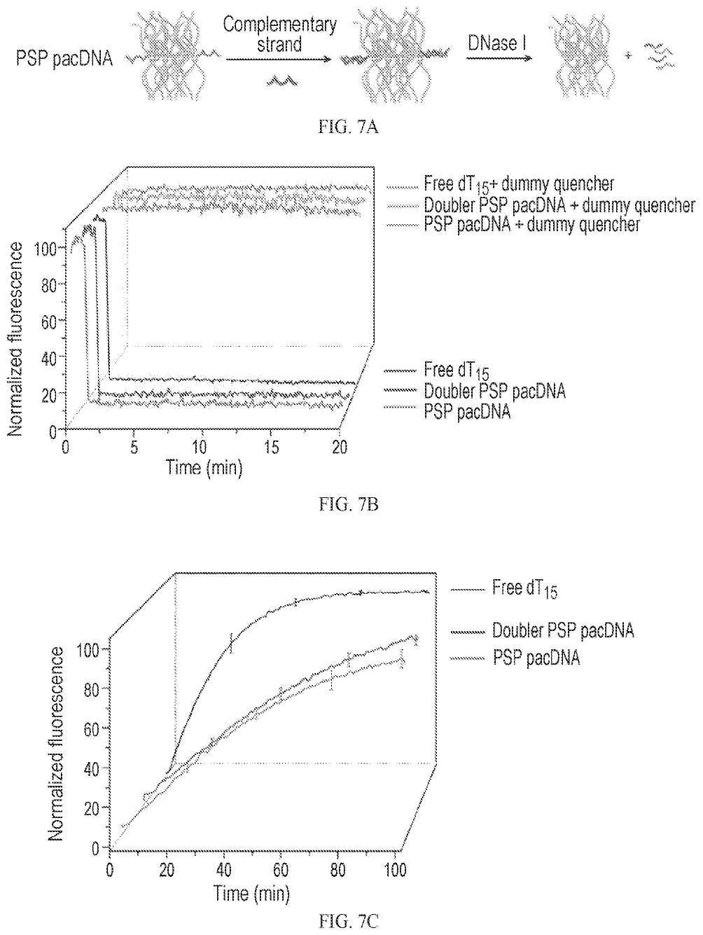

The PSP pacDNAs are designed to reduce unwanted oligonucleotide-protein interactions, inhibit non-specific cellular uptake, and prolong blood circulation times. To test the first of these characteristics, the nuclease degradation kinetics of various PSP pacDNAs were examined (FIG. 7A). The 3′ of dT₁₅ was labeled with the fluorophore Cy5, and a 5′ quencher (dabcyl)-labeled complementary dA₁₅ strand was hybridized to the PSP pacDNAs. The hybridization kinetics for all PSP pacDNAs were indistinguishable from that of free dT₁₅, reaching completion in ≈10 s. The addition of a non-complementary, quencher-labeled dummy strand did not result in a reduction in Cy5 fluorescence (FIG. 7B), ruling out non-specific binding. Upon digestion by the endonuclease DNase I, the fluorophore-quencher pair is separated, leading to an increase of fluorescence. The degradation half-lives of the PSP pacDNAs are roughly three times that of free double-stranded DNA, suggesting some steric shielding effects (FIG. 7C and Table 6). In contrast, PEGylation by conventional linear or slightly branched PEG does not alter the degradation half-life by DNase I.

TABLE 6
Enzymatic half-lives of free dT₁₅ and PSP pacDNAs

| Sample ID | t_{1/2} (min) |
|---|---|
| Free dT₁₅ | 13.93(±0.53) |
| PSP pacDNA | 32.18(±6.35) |
| Doubler PSP pacDNA | 38.15(±1.79) |

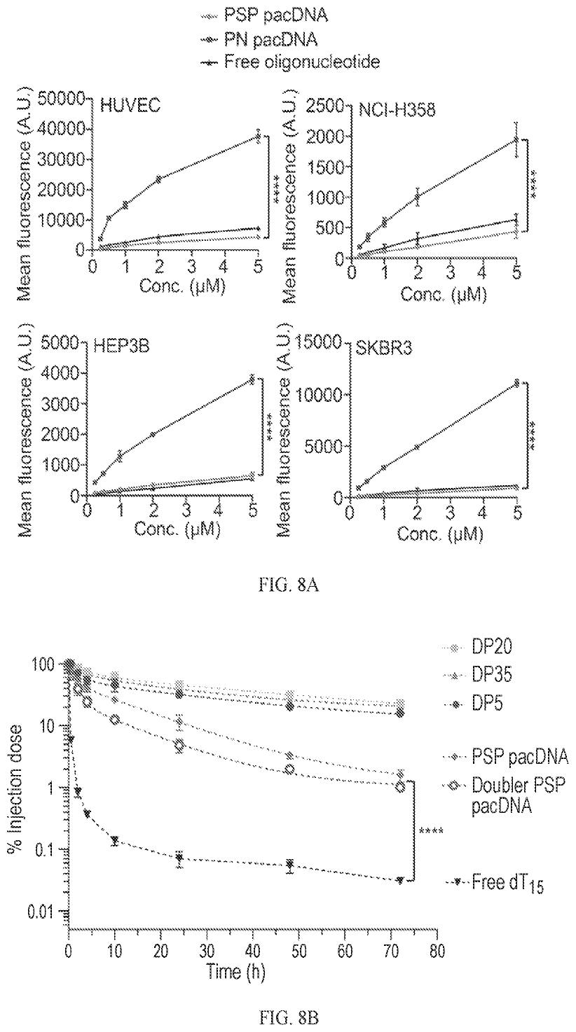

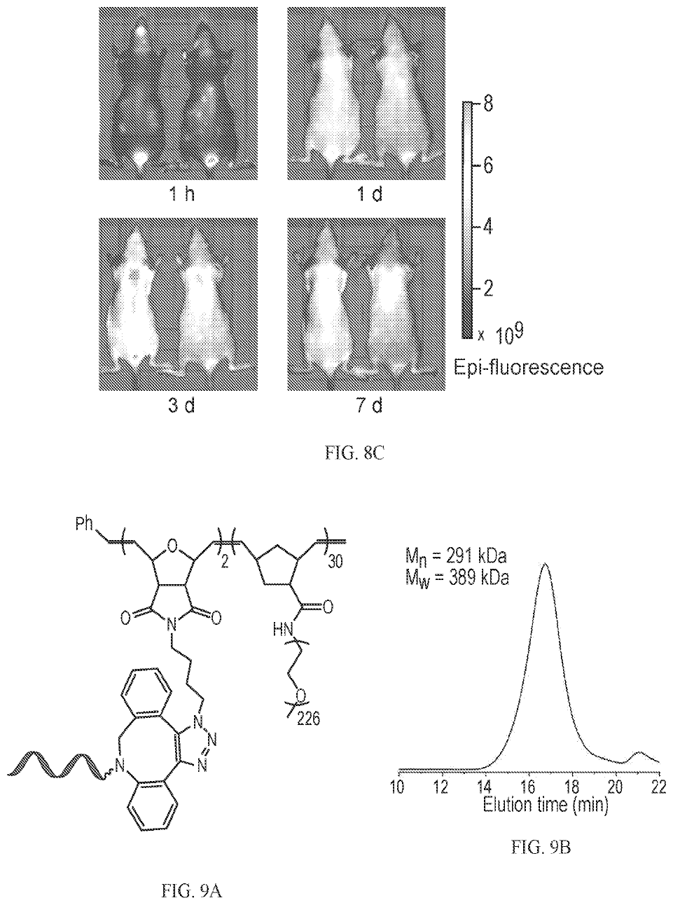

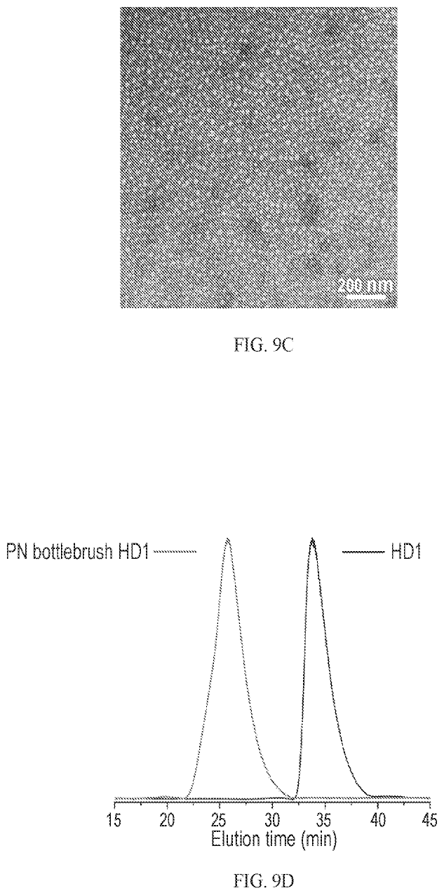

It is speculated that a mechanism for the brush-type PEG to enter the cell involves transient adsorption of the polymer onto the plasma membrane (PM), possibly mediated by PEG-cation interactions and the negative PM potential. With PN-based bottlebrushes, the near-neutral ζ potential promotes PM adsorption, and the hydrophobic polymer backbone then further increases adhesion strength and therefore polymer residence times on the PM, allowing for increased uptake compared with normal PEG or DNA. In contrast, the more negative ζ potential and the completely hydrophilic backbone of the PSP pacDNA should reduce the transient polymer-PM interactions and therefore endocytosis. To test this hypothesis, the cellular uptake of a Cy3-labeled PSP pacDNA and a PN-based counterpart by HUVEC (endothelial), NCI-H358 (lung), HEP3B (liver), and SKBR3 (breast) cells using flow cytometry were compared. The results show that the PSP pacDNA consistently undergoes very limited uptake, similar to the levels exhibited by free single-stranded DNA which has been known to exhibit insignificant levels of endocytosis (FIG. 8A). Conversely, the PN pacDNA, which has similar MW, architecture, and PEG/DNA content as the PSP pacDNA (FIG. 9A, 9B, 9C, 9D), shows 6-12 times more rapid cell uptake than PSP pacDNA. These results demonstrate that a small change in the chemical composition of the backbone (no more than 5% in overall MW) can greatly alter the biological characteristics of the bottlebrush polymer.

Persistence in plasma is important to aptamers as rapid clearance can significantly shorten the duration of effect and increase dosage requirements. It is hypothesized that PSP pacDNA can improve the plasma PK and the potency of conjugated aptamers by reducing cell uptake levels and avoiding renal clearance. To evaluate the plasma PK, 5′-Cy5-labeled PSP bottlebrushes, PSP pacDNA, and doubler PSP pacDNA were injected into C57BL/6 mice through the tail vein. Blood samples were collected from the submandibular vein at predetermined time points and analyzed using a two-compartment model. Remarkably, the PSP bottlebrushes exhibited very high stability and retention in plasma, with distribution half-lives in the range of 1.6-2.6 h and elimination half-lives between 24 and 35 h (Table 6). There was still >20% of the injected dose (for DPs=20 and 35) remaining in circulation after 72 h (FIG. 8B). For the PSP pacDNA, the plasma clearance is notably faster than the bottlebrushes alone, likely due to degradation of the dT₁₅ component by plasma nucleases. The doubler PSP pacDNA was cleared slightly faster than the linear counterpart. Still, >10% of both PSP pacDNAs was found within the plasma 24 h post-injection, while there was <1% free dT₁₅ in circulation after only 2 h and <0.1% after 24 h. Estimating bioavailability by calculating the area under the curve (AUC_{∞}), PSP pacDNA are 15-25 times more bioavailable than free dT₁₅ (Table 7).

TABLE 7
Plasma pharmacokinetics parameters of free dT₁₅, PSP
bottlebrushes, and PSP pacDNAs in C57BL/6 mice.

| Sample ID | t_{1/2α} (h) | t_{1/2β} (h) | AUC_{∞} (nmol/mL*h) |
|---|---|---|---|
| Free dT₁₅ | 0.26 | 0.51 | 2.68 |
| PSP pacDNA | 1.06 | 10.80 | 67.79 |
| Doubler PSP pacDNA | 1.02 | 9.47 | 38.96 |
| DP5 | 1.61 | 24.77 | 190.60 |
| DP20 | 2.63 | 35.34 | 310.56 |
| DP35 | 2.06 | 24.09 | 314.04 |

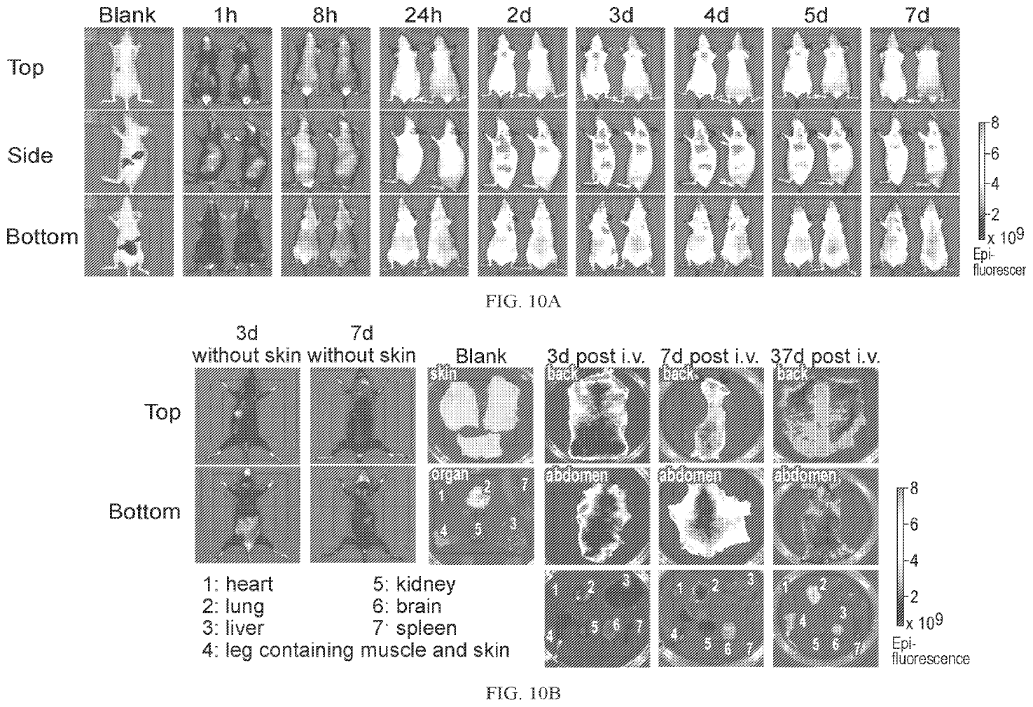

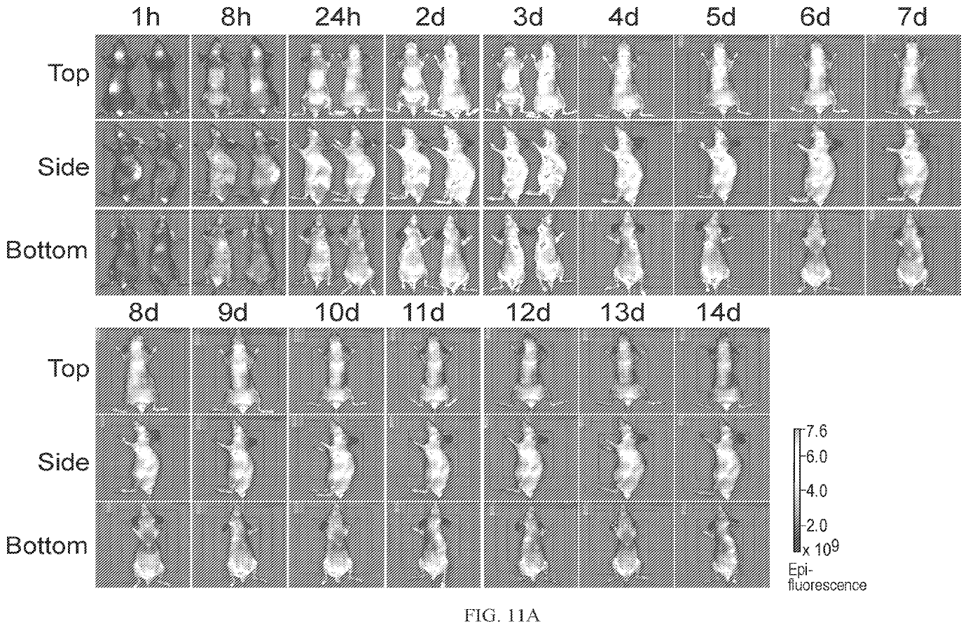

To follow the bottlebrush component of the PSP pacDNA in vivo for an extended period of time and investigate its biodistribution, SKH1-Elite mice, an immunocompetent hairless strain, were injected with a 5′-Cy5-labeled PSP bottlebrush with a DP of 30 via the tail vein. Live animal imaging showed that fluorescence gradually increased near the skin of the mice, reaching peak levels after 48 h. The fluorescence persisted at peak levels until approximately the 7th day post injection (FIG. 8C and FIG. 10A, 10B), before slowly decreasing to slightly above-background levels in five weeks. On days 3, 7, and 37, animals were euthanized, and major organs were excised for imaging. All major internal organs (bar the brain) as well as muscle exhibited uptake without a strong preference for any organ. However, interestingly, pronounced accumulation in the skin (including epidermis, dermis, and the skin-draining lymph nodes) was observed. The skin was also the only organ to continue to exhibit fluorescence on day 37. In immunodeficient athymic nude mice, which lack T cells, a similar observation was made (FIG. 11A, 11B). The skin accumulation of PSP pacDNA may be due to the characteristics of the terminal networks of blood supply capillaries and/or the eventual uptake of the PSP pacDNA by skin-resident immune cells, such as dendritic cells, macrophages, and mast cells. Additional studies are required to elucidate the underlying mechanism for this phenomenon.

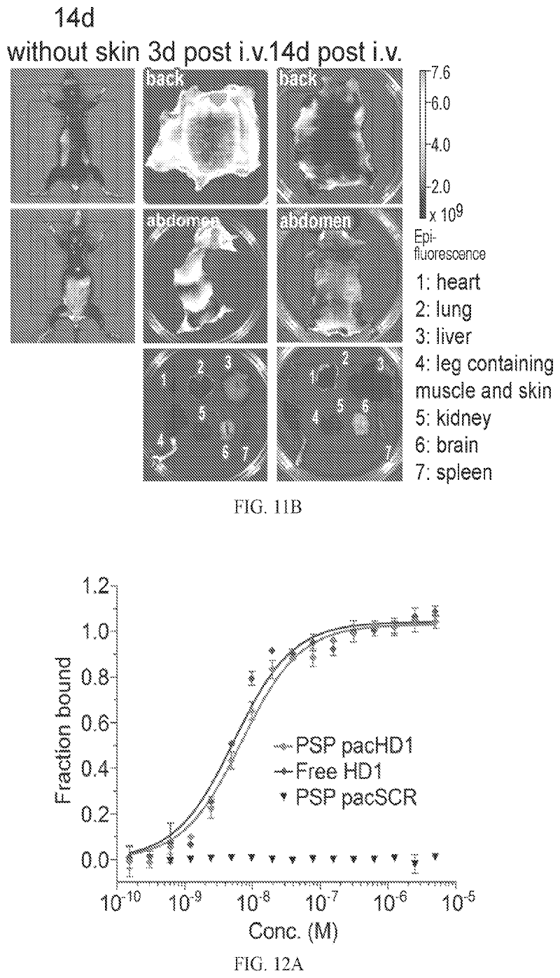

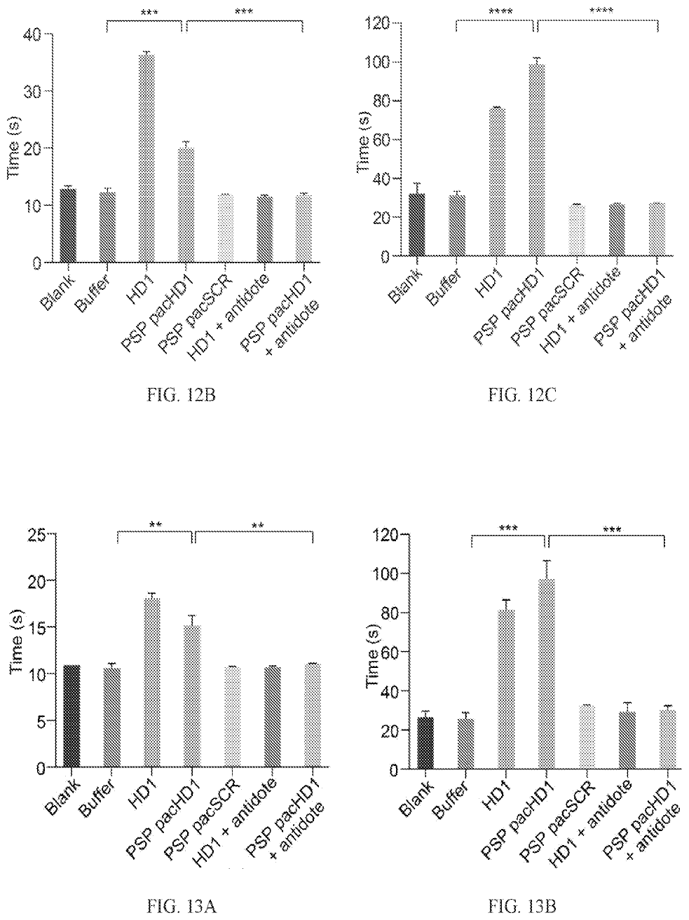

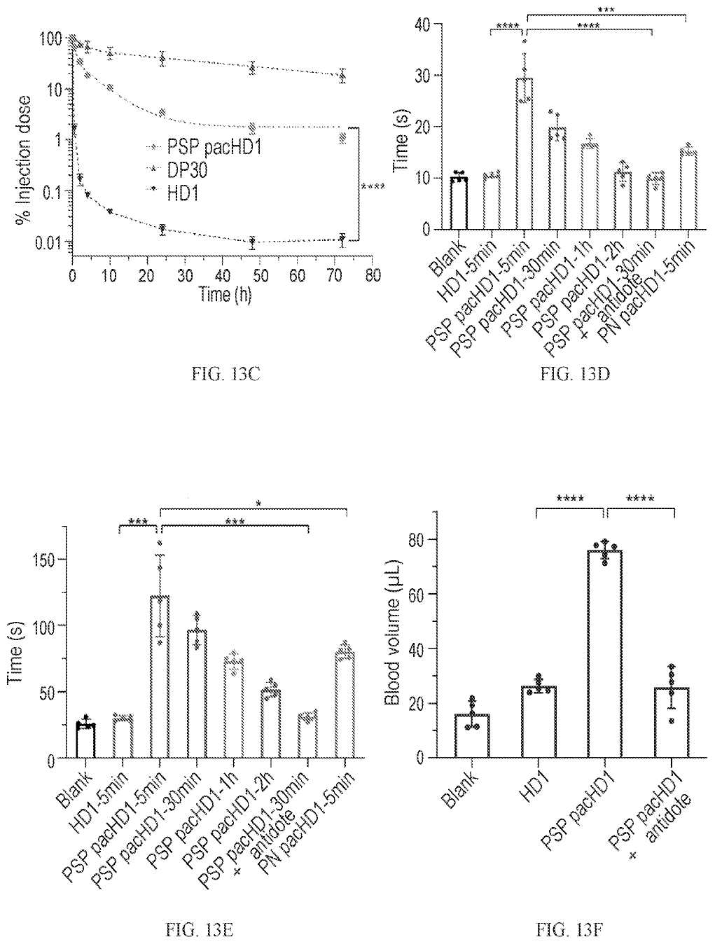

To demonstrate the binding efficacy of PSP pacDNA containing a functional aptamer, a thrombin-binding aptamer, HD1, was adopted as a model system. HD1 is a 15-nucleotide DNA sequence folding into an antiparallel Gquadruplex that can specifically bind to the exosite I of human alpha thrombin. Upon binding, HD1 can inhibit the coagulation and prolong coagulating times. Having a dissociation constant (k_{d}) in the nanomolar range, HD1 has been envisioned as a short-term anticoagulant that can be used intraoperatively to reduce the risk of thromboembolism. Several studies have demonstrated improved ex vivo efficacy of HD1 through polymeric micelles or DNA origami (Roloff, Alexander, et al. “Micellar thrombin-binding aptamers: reversible nanoscale anticoagulants.” Journal of the American Chemical Society 139.46 (2017); Zhao, Shuai, et al. “A DNA origami-based aptamer nanoarray for potent and reversible anticoagulation in hemodialysis.” Nature Communications 12.1 (2021); the contents of which are incorporated herein in their entirety). However, in vivo potency of HD1 has not been robustly demonstrated. The binding affinity of HD1 and the corresponding pacDNA (PSP pacHD1) were assessed by microscale thermophoresis (MST) using Cy5-labeled human alpha thrombin as the target. It was found that the appendage of the bottlebrush structure to HD1 has a nominal impact on its binding affinity, with the kd of free HD1 and PSP pacHD1 being 5.4 nM and 6.9 nM, respectively. In addition, a PSP pacDNA containing a scrambled sequence (PSP pacSCR) exhibited no measurable binding with thrombin, ruling out the bottlebrush component being responsible for the observed binding (FIG. 12A). Next, the anticoagulation properties of HD1 and PSP pacHD1 in human plasma by measuring the prothrombin time (PT) and activated partial thromboplastin time (aPTT) were evaluated (FIGS. 12B and 12C). Interestingly, while free HD1 induced a more pronounced effect in the PT assay, the PSP pacHD1 was more effective in the aPTT assay, with almost three-fold longer coagulating times compared to the vehicle. One interpretation for this difference is that the intrinsic/extrinsic pathways of the coagulation cascade are affected differently by the test agents. Again, PSP pacSCR did not show anticoagulation effects. Importantly, the anticoagulation was completely reversible through the use of a locked nucleic acid (LNA)-based antidote that consists of a fully complementary strand to HD1, which disrupts its secondary structure. Such an antidote would be an invaluable tool for the perioperative management of patients receiving anticoagulants. Interestingly, although developed as a human thrombin binder, HD1 can also cause prolonged coagulation in mouse plasma, and the strengths of the effect were found to be comparable in both species. Furthermore, the diverging relative strengths of HD1 and PSP pacHD1 in PT and aPTT assays were also observed in mouse plasma (FIGS. 13A and 13B).

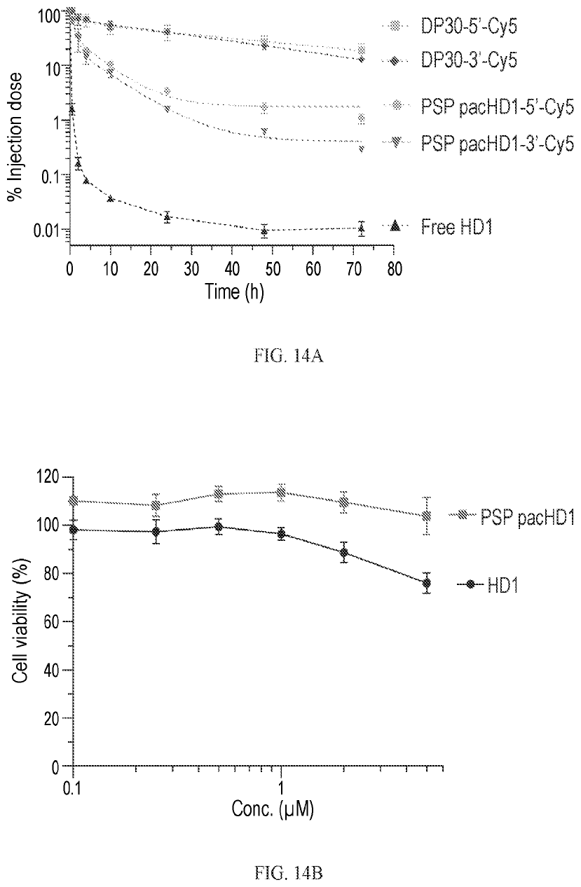

The complex and dynamic in vivo environment represents the ultimate challenge for aptamers. To assess the ability of the PSP pacDNA to serve as an aptamer enhancer in vivo, free HD1 and the PSP pacHD1 were injected in vivo in C57BL/6 mice. Plasma PK measurements of 5′-Cy5-labeled materials reveal that the PSP pacHD1 persists in the blood markedly longer than free HD1, with a 16-fold difference in AUC_{∞} (FIG. 13C). Interestingly, placing the Cy5 at the 5′ of PSP pacDNA results in slightly better blood retention than placement at the 3′ (see FIG. 14A and Table 8 for PK parameters).

TABLE 8
Calculated parameters of 3′- and 5′-Cy5-labeled PSP bottlebrush (DP30-
5′-Cy5 and DP30-3′-Cy5) and PSP pacHD1 in C57BL/6 mice

| Sample ID | t_{1/2α} (h) | t_{1/2β} (h) | AUC_{∞} (nmol/mL*h) |
|---|---|---|---|
| Free HD1 | 0.25 | 0.43 | 2.12 |
| DP30-5′-Cy5 | 2.13 | 47.56 | 204.4 |
| DP30-3′-Cy5 | 1.21 | 31.86 | 188.7 |
| PSP pacHD1-5′-Cy5 | 0.79 | 5.94 | 34.1 |
| PSP pacHD1-3′-Cy5 | 0.56 | 4.76 | 24.1 |

The difference may be due to the fact oligonucleotide degradation in plasma (both mouse and human) occurs primarily by 3′-to-5′ exonucleolytic activity. When analyzing anticoagulating properties, it was found that free HD1 produced no statistically significant difference compared to blank in both PT and aPTT assays using blood collected 5 min after injection, although in purified plasma HD1 does exhibit potency (see above). The rapid and complete loss of activity of free HD1 in vivo may be attributed to a combination of degradation, non-specific binding, and rapid renal clearance. In stark contrast, the PSP pacHD1 exhibited a 3-fold increase in PT and a 5-fold increase in aPTT measurements relative to blank (FIGS. 13D and 13E). The effect attenuates slowly, reaching baseline levels in ≈2 h. When a dose of the LNA antidote (10 equiv to HD1) was delivered intravenously 20 min after the administration of PSP pacHD1, the anticoagulating effect was completely neutralized in the next blood collection timepoint (30 min). Comparing the PSP pacHD1 to its PN-based counterpart, it can be seen that the PN pacHD1 produced <50% of the effect as the PSP pacHD1 at the 5 min timepoint, although both types of polymers exhibit similar plasma PK, which highlights the importance of the phosphodiester backbone of the PSP pacDNA. The in vivo anticoagulatory effect of PSP pacHD1 was further quantified using a murine tail-transection bleeding model (FIG. 13F). Shortly (5 min) after receiving the test agents and controls, the tails of mice were clipped and blood from the tail was collected for 15 min. Treatment with PSP pacHD1 induced the largest amount of blood loss (76 μL), while free HD1- and vehicle-treated mice lost 26 μL and 16 μL blood, respectively. When mice were given PSP pacHD1 and the LNA antidote sequentially, tail bleeding reverted to the baseline rate. Taken together, these results demonstrate that the PSP pacDNA structure considerably enhances the plasma PK, bioavailability, and the potency of the conjugated aptamer, and the activity is controllable using an antidote.

The PSP pacHD1 was found to be non-cytotoxic up to 5 μM for NCI-H358 cells (FIG. 14B). However, free HD1 exhibits slight inhibition of cell growth, possibly due to nonspecific binding to cell surface receptors leading to erroneous signaling. Activation of the immune system by PSP pacHD1 was investigated by analyzing complement C3 and selected cytokines in C57BL/6 mice following systemic delivery. The treatment with PSP pacHD1, PN pacHD1, and free HD1 resulted in no significant change in C3 levels relative to PBS control (FIG. 14C). Tumor necrosis factor-α (TNF-α) and interleukin 6 (IL-6) exhibited statistically significant activation for the PSP pacHD1 group compared to PBS control, but the activation is weak. IL-12 levels showed no obvious changes. In contrast, lipopolysaccharide (LPS, positive control), induced strong expression of all three cytokines. These results are consistent with previous findings that the bottlebrush architecture makes the conjugate uniquely non-immunogenic (even for the PEG component), while the conventional, linear PEG-DNA conjugate can elicit both innate and adaptive immune responses. Collectively, these data suggest that the PSP pacHD1 is generally safe without major acute toxic or immunological shortcomings.

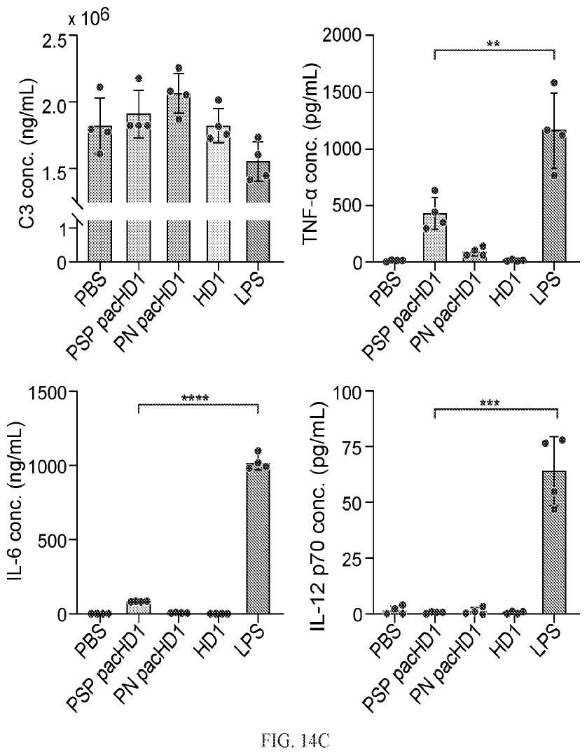

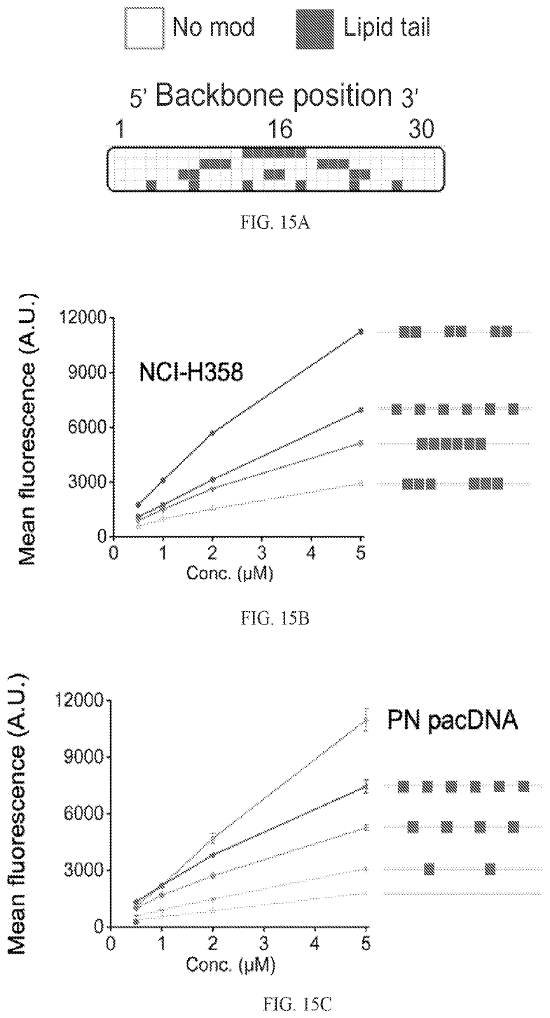

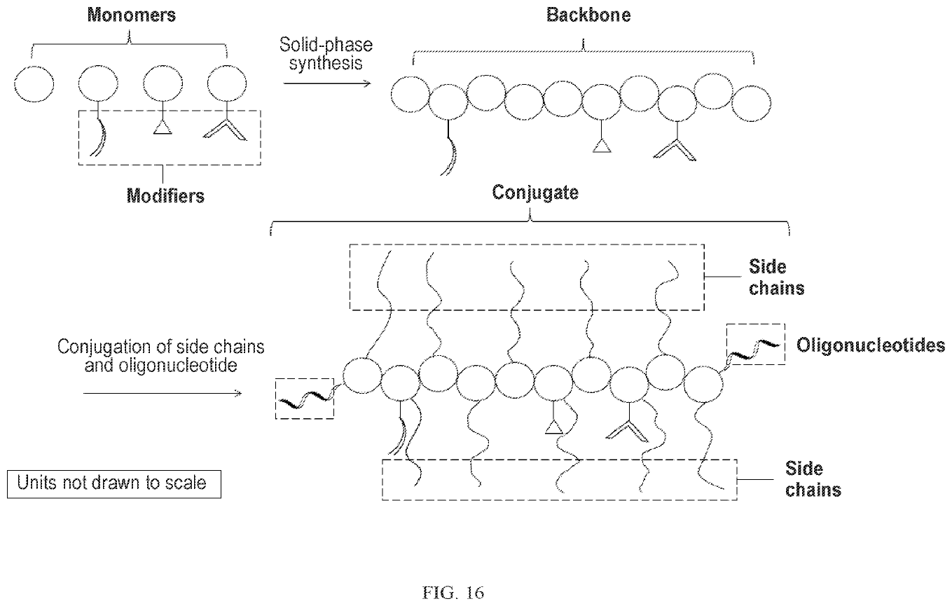

To demonstrate how the backbone sequence can be used to alter biological properties of the pacDNA, a series of pacDNA with 0-6 lipid modifiers evenly distributed into backbone and a second series each containing six lipid modifiers in the backbone but with varying grouping densities were synthesized (FIG. 15A). Dynamic light scattering (DLS) and size exclusion chromatography (SEC) reveal uniform sizes and no aggregation for all samples. It can be clearly seen that the cell uptake by NCI-H358 cells increases almost linearly with the number of lipid tails (FIG. 15B). Remarkably, various samples containing the same number of lipid tails (six) differ ˜5-fold in cell uptake despite being compositionally identical, with the highest uptake level matching that of the PN-based pacDNA (FIG. 15C). These results demonstrate that the presence and arrangement of the modifiers within the pacDNA backbone can have a significant impact on the materials-cell interactions, and by screening a library of pacDNA with variable backbone sequences and compositions, it is possible to identify sequences that provide optimal biological properties.

### CONCLUSION

In summary, the present disclosure provides for a facile route to a novel bottlebrush polymer that can be used to enhance the pharmacological properties and in vivo performance of oligonucleotides. These unimolecular polymer-oligonucleotide conjugates can be synthesized in the same step as the therapeutic sequence via the automated solid-phase methodology, followed by near-quantitative PEGylation. The synthesis is highly versatile with regard to the size, backbone sequence, and architecture of the final polymer and does not involve heavy metal catalysts that can complicate downstream applications. Importantly, the PSP pacDNA consists only of building blocks that are recognized as safe in pharmaceutical applications, and does not involve a non-degradable, long-chain aliphatic backbone. Astonishingly, these results show that the pacDNA (i.e., conjugate) lacks cellular uptake by non-phagocytic cells but exhibits superior plasma retention. The phosphodiester backbone of the PSP pacDNA can be designed to either resist or promote cellular uptake, and the spatially congested PEG environment reduces nonspecific binding, leading to elevated blood retention times and increased productive binding. These properties impart the PSP pacDNA superior performance in an anticoagulation mouse model compared to the free aptamer, which exhibits potency in vitro but no activity in vivo. Importantly, the sequence-defined backbone makes it possible to screen for pacDNAs with specific biological properties from a library of pacDNAs, yielding optimal structures on a disease-specific level in therapeutics delivery.

### Experimental Procedures

### Materials

Methoxy polyethylene glycol (PEG) glutaramide succinimidyl ester (Mn=10 kDa) was purchased from Creative PEGWorks (Chapel Hill, NC, USA). Phosphoramidites and supplies for DNA synthesis were purchased from Glen Research Co. (Sterling, VA, USA). Human NCI-H358 lung cancer cell line, human SKBR3 breast cancer cell line, human Hep3B liver cancer cell line and primary human umbilical vein endothelial cell line (HUVEC) were purchased from American Type Culture Collection (Rockville, MD, USA). Human pooled normal plasma was purchased from George King Bio-Medical, Inc. (Overland Park, KS, USA). All other materials were purchased from Fisher Scientific Inc. (USA), Sigma-Aldrich Co. (USA), or VWR International LLC. (USA), and used as received unless otherwise indicated.

### Methods

¹H nuclear magnetic resonance (NMR) spectra were recorded on a Varian 500 MHz NMR spectrometer (Varian Inc., CA, USA). MALDI-TOF mass spectrometry (MS) measurements were performed on a Bruker Microflex LT mass spectrometer (Bruker Daltonics Inc., MA, USA). Concentrations of samples were determined using a Nanodrop™ 2000 spectrophotometer (Thermo Scientific, USA) and a BioTek Synergy Neo2 Hybrid Multi-Mode Reader (BioTek Instruments Inc., VT, USA). DLS and ζ potential measurements were performed on a Malvern Zetasizer Nano-ZSP (Malvern, UK). Samples were dissolved in Nanopure™ water at a concentration of 1 μM and filtered through a 0.2 μm PTFE filter before measurement. Fluorescence spectroscopy was carried out on a Cary Eclipse fluorescence spectrophotometer (Varian Inc., CA, USA). Reversed-phase high-performance liquid chromatography (RP-HPLC) was performed on a Waters (Waters Co., MA, USA) Breeze 2 HPLC system coupled to a Symmetry® C18 3.5 μm, 4.6×75 mm reversed-phase column and a 2998 PDA detector, using TEAA buffer (0.1 M) and HPLC-grade acetonitrile as mobile phases. Aqueous gel permeation chromatography (GPC) analysis was carried out on a Waters Breeze 2 GPC system equipped with a series of an Ultrahydrogel™ 1000, 7.8×300 mm column and three Ultrahydrogel™ 250, 7.8×300 mm columns and a 2998 PDA detector. Sodium nitrate solution (0.1 M) was used as the eluent running at a flow rate of 0.8 mL/min. The number- and weight-average molecular weights of a polymer sample were calculated based upon sodium polystyrene sulfonate (PSSNa) calibration standards with a MW range of 1,600 to 2,500 kDa (Scientific Polymer Products Inc., New York, USA), while polydispersity indices (PDIs) were determined using PAGE-purified dT₁₅ DNA oligomer as standard, assuming the oligomer has a PDI of 1.01. N,N-dimethylformamide (DMF) GPC was performed on a Tosoh EcoSEC HLC-8320 GPC system (Tokyo, Japan) equipped with a TSKGel α-M 7.8×300 mm, 13 μm column and RI/UV-Vis detectors. HPLC-grade DMF with 0.05 M lithium bromide was used as the mobile phase, and samples were analyzed at a flow rate of 0.4 mL/min. DMF-GPC calibration was based on a ReadyCal kit of polyethylene glycol standards (PSSPolymer Standard Service-USA Inc., MA, USA). The kit covers an Mn range from 232 Da to 1015 kDa. For atomic force microscopy (AFM) measurements, samples were dissolved in Nanopure™ water and diluted to a concentration of 1 μM. 10 μL of each sample was placed onto freshly cleaved mica (Ted Pella Inc., CA, USA) and allowed for drying. All the samples were imaged on a Dimension FastScan AFM (Bruker Corporation, USA), under the ScanAsyst in Air mode. SCANASYST-Fluid+ probes (Bruker Corporation, USA) were used for all samples. For transmission electron microscopy (TEM), samples (10 μM) were placed on parafilm as a droplet, onto which a copper-coated TEM grid was gently placed. The grids were then moved, dried, and stained using 2% uranyl acetate for 10 min. TEM images were collected on a JEOL JEM 1010 electron microscope with an accelerating voltage of 80 kV.

### Oligonucleotide and Poly(Serinol Phosphodiester) (PSP) Backbone Synthesis

All oligonucleotides and PSP backbones were synthesized on a Model 391 DNA synthesizer (Applied Biosystems, Inc., Foster City, CA). The time for the coupling step of the serinol-phosphoramidite was set at 10 min (compared to 15 s for normal phosphoramidites). Oligonucleotide strands were cleaved from the CPG support and deprotected in aqueous ammonium hydroxide solution (28-30% NH₃ basis) at room temperature for 24 h. PSP backbones containing Fmoc-serinol units were deprotected on-column in DMF with 20% piperidine 3× and washed with DMF 2×. The CPG was dried in vacuo and cleaved via the same method as normal oligonucleotide strands. All PSP backbones and oligonucleotide strands were purified by RP-HPLC, followed by the removal of dimethoxytrityl (DMT) groups using 20% acetic acid.

### Synthesis of Serinol-Phosphoramidite

### Synthesis of Fmoc-β-Ala-Serinol

Fmoc-β-alanine (1) (9.33 g, 30 mmol) and N-hydroxysuccinimide (3) (3.45 g, 30 mmol) were dissolved in a mixed solvent of dichloromethane (DCM, 120 mL) and DMF (6 mL). N,N′-dicyclohexylcarbodiimide (3) (DCC, 6.18 g, 30 mmol) was dissolved in DCM (12 mL) and added to the mixture. The mixture was stirred at room temperature for 1.5 h, and a white precipitate of N,N′-dicyclohexylurea (DCU) was observed.

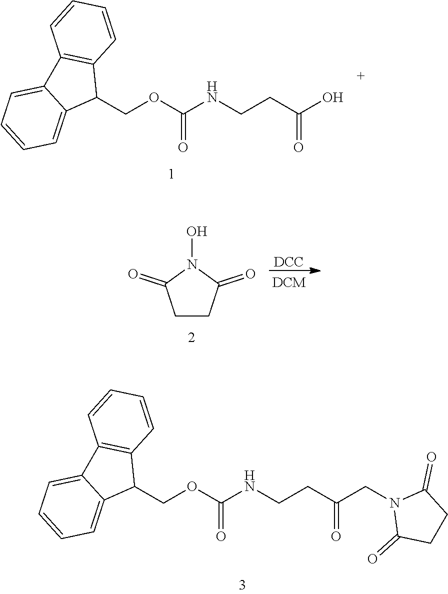

The reaction mixture was filtered, and the liquid was transferred to a mixture containing serinol (2.73 g, 30 mmol) and pyridine (42 mL). The reaction mixture was allowed to stir for 16 h at room temperature, before the solvent was removed by evaporation to yield a semi-solid residue (containing residue pyridine). Toluene (50 mL) was added to the residue and co-evaporated under reduced pressure 3× to remove pyridine. The resulting white solid was refluxed in DCM (200 mL) for 2 h, before being chilled to −20° C. The product was then collected by filtration and washed with DCM (100 mL) 2× and diethyl ether (100 mL) 1×. The final product was dried under high vacuum and stored at −20° C. The isolated yield for 4 is 75±5%. ¹H NMR (500 MHz, DMSO-d₆) δ 7.89 (d, J=7.6 Hz, 2H), 7.68 (d, J=7.5 Hz, 2H), 7.56 (d, J=8.2 Hz, 1H), 7.42 (t, J=7.5 Hz, 2H), 7.33 (t, J=7.4 Hz, 2H), 7.27 (t, J=5.9 Hz, 1H), 4.58 (t, J=5.7 Hz, 2H), 4.27 (d, J=6.7 Hz, 2H), 4.20 (t, J=7.1 Hz, 1H), 3.70 (q, J=6.3 Hz, 1H), 3.38 (t, J=5.4 Hz, 4H), 3.18 (q, J=6.9 Hz, 2H), 2.27 (t, J=7.3 Hz, 2H) (FIG. 2A); Maldi-TOF: m/z calculated for C₂₁H₂₄N₂O₅ [M-H]⁻ 383.17, found 383.70.

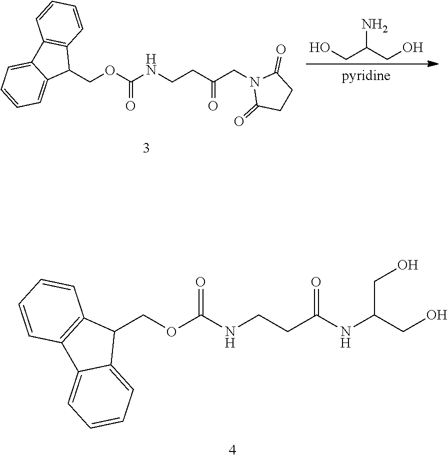 [SMILES: O=C(CCNC(=O)OCC1c2ccccc2-c2ccccc21)CN1C(=O)CCC1=O ; NC(CO)CO ; O=C(CCNC(=O)OCC1c2ccccc2-c2ccccc21)NC(CO)CO]

### Synthesis of Fmoc-β-Ala-Serinol-DMT

Fmoc-β-Ala-serinol (4) (1, 5.88 g, 15.3 mmol) was placed in a flask filled with N₂, and was dissolved in pyridine (16 mL). The flask was chilled in an ice bath. 4,4′-DMT chloride (5) (5.37 g, 15.9 mmol) was dissolved in pyridine (32 mL) and added dropwise to the mixture containing molecule (4). The mixture was allowed to stir under N₂ for 1 h in an ice bath and then overnight at room temperature. Methanol (1 mL) was added to the mixture and stirred for 15 min to quench the reaction. Removal of the solvent under reduced pressure yields an oily residue, which was then co-evaporated with toluene (40 mL) 3×. The mixture was dissolved in DCM (50 mL) and washed with 5% sodium bicarbonate solution (50 mL) and then with brine (50 mL), before being dried over anhydrous sodium sulfate. The crude product was concentrated and purified by silica gel column purification (ethyl acetate/methanol/triethylamine 95:5:1 v:v:v). The isolated yield of 6 is 43±5%. ¹H NMR (500 MHz, Chloroform-d) δ 7.75 (d, J=7.5 Hz, 2H), 7.57 (dd, J=7.4, 2.5 Hz, 2H), 7.39 (d, J=7.6 Hz, 4H), 7.33-7.24 (m, 9H), 6.83 (d, J=8.4 Hz, 4H), 5.99 (d, J=7.8 Hz, 1H), 5.53 (d, J=6.5 Hz, 1H), 4.34 (dd, J=7.3, 3.7 Hz, 2H), 4.23-4.14 (m, 1H), 3.81 (dd, J=11.4, 4.8 Hz, 1H), 3.76 (s, 7H), 3.69 (dd, J=11.3, 4.3 Hz, 1H), 3.48 (p, J=7.3 Hz, 2H), 3.39-3.25 (m, 2H), 2.39 (q, J=6.0 Hz, 2H) (FIG. 2B); Maldi-TOF: m/z calculated for C₄₂H₄₂N₂O₇ [M+Na]⁺ 709.29, found 709.17.

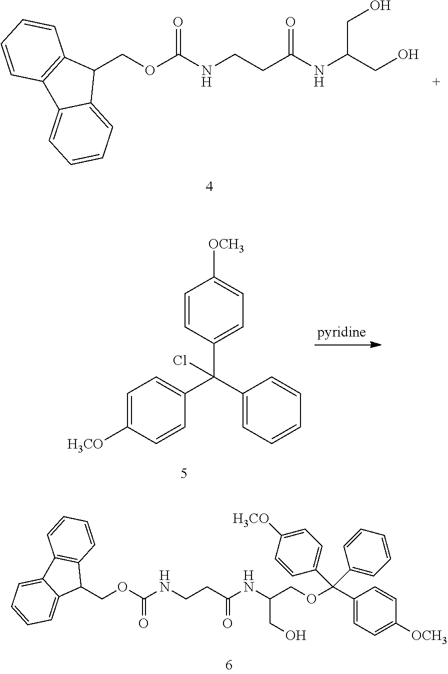 [SMILES: O=C(CCNC(=O)OCC1c2ccccc2-c2ccccc21)NC(CO)CO ; COc1ccc(C(Cl)(c2ccccc2)c2ccc(OC)cc2)cc1 ; COc1ccc(C(OCC(CO)NC(=O)CCNC(=O)OCC2c3ccccc3-c3ccccc32)(c2ccccc2)c2ccc(OC)cc2)cc1]

### Synthesis of Serinol-Phosphoramidite

Fmoc-β-Ala-serinol-DMT (6) (2.76 g, 4 mmol) was placed in a flask charged with N₂ and dissolved in dry DCM (12 mL) containing N,N-diisopropylethylamine (DIPEA, 3.5 mL). The flask was chilled in an ice bath. 2-Cyanothyl-N,N-iisopropylchlorophosphoramidite (7) (1.9 g, 8 mmol) was dissolved in DCM (4 mL) and added dropwise to the mixture containing 6. The reaction mixture was allowed to stir vigorously for 20 min before being warmed to room temperature and then stirred for another 40 min. An excess of ethyl acetate (EA) was added to the reaction mixture. The mixture was washed with saturated sodium bicarbonate solution (10 mL). After drying over anhydrous sodium sulfate, the mixture was filtered, and the filtrate was concentrated for silica gel column purification (hexane/ethyl acetate/triethylamine 67:33:1 v:v:v). The isolated yield of 8 is 66±5%. ¹H NMR (500 MHz, Chloroform-d) δ 7.77 (d, J=7.5 Hz, 2H), 7.59 (dd, J=7.6, 4.1 Hz, 2H), 7.46-7.37 (m, 4H), 7.32 (d, J=7.8 Hz, 8H), 7.23 (d, J=7.4 Hz, 1H), 6.84 (d, J=8.2 Hz, 4H), 5.84 (d, J=8.6 Hz, 1H), 5.57 (q, J=6.7 Hz, 1H), 4.34 (d, J=7.4 Hz, 2H), 4.16 (dt, J=29.2, 7.3 Hz, 1H), 3.80 (d, J=11.5 Hz, 9H), 3.71-3.62 (m, 2H), 3.61-3.51 (m, 2H), 3.49 (s, 2H), 3.42-3.28 (m, 1H), 3.16 (dt, J=9.3, 6.2 Hz, 1H), 2.60-2.47 (m, 2H), 2.38 (dd, J=13.3, 6.7 Hz, 2H), 1.20-1.11 (m, 12H) (FIG. 2C).

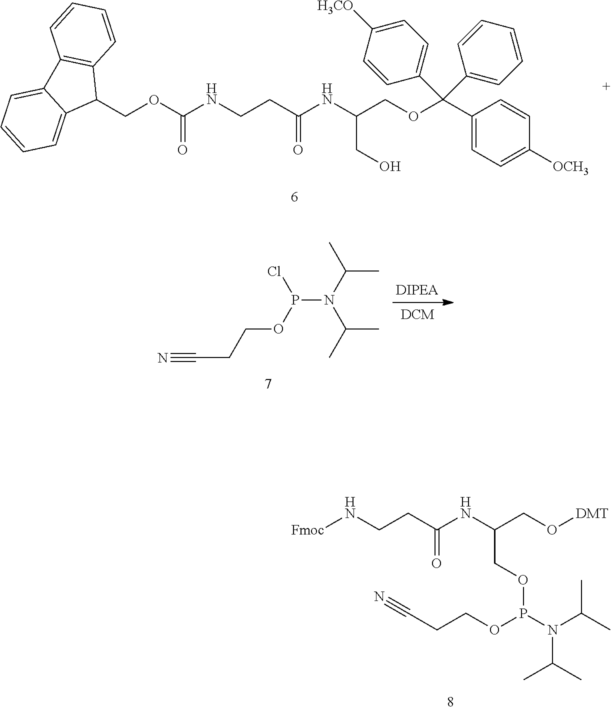

Synthesis of PN pacDNA

Norbornenyl bromide (9) and norbornenyl PEG (10) were synthesized as previously described in Lu, et al. “Effective antisense gene regulation via noncationic, polyethylene glycol brushes.” Journal of the American Chemical Society 138 (2016); the contents of which are incorporated herein by reference in their entirety.

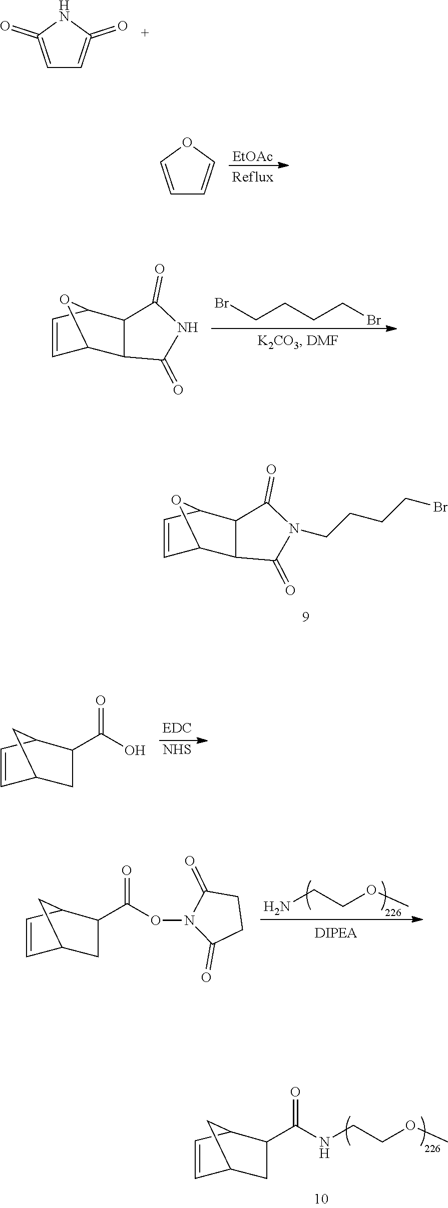

Modified 2nd generation Grubbs catalyst was prepared based on a published method shortly prior to use. Next, norbornenyl bromide (9) (5 equiv.) was dissolved in deoxygenated DCM under N2 and cooled to −20° C. in an ice-salt bath. The modified Grubbs' catalyst (1 equiv.) in deoxygenated DCM was added to the solution via a gastight syringe, and the solution was stirred vigorously for 30 min. After thin-layer chromatography (TLC) confirmed the complete consumption of the monomer, norbornenyl PEG (50 equiv.) in deoxygenated DCM was added to the reaction, and the mixture was stirred for 6 h. Several drops of ethyl vinyl ether were added to quench the reaction and the solution was stirred for an additional 2 h. After concentration under vacuum, the residue was precipitated into cold diethyl ether 3×. The precipitant was dried under vacuum to afford a white powder.

Subsequently, the PN-backboned brush polymer was reacted with an excess of sodium azide in anhydrous DMF overnight at room temperature. The materials were transferred to a dialysis tubing (MWCO, 10 kDa), dialyzed against Nanopure™ water for 24 h, and lyophilized to afford a white powder.

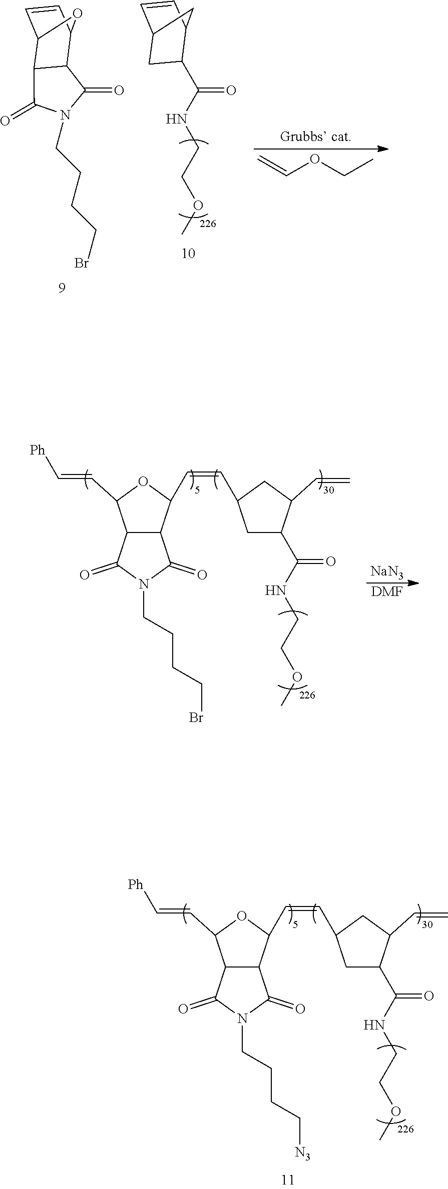

The azide-functionalized PN bottlebrush polymer (11) (50 nmol) was dissolved in 1 mL sodium chloride solution (3 M) and reacted with dibenzocyclooctyne (DBCO)-modified oligonucleotides (100 nmol) at 50° C. overnight. The conjugate was purified by aqueous GPC, desalted, and lyophilized. The purified PN pacDNA (12) were stored at −20° C. before use.

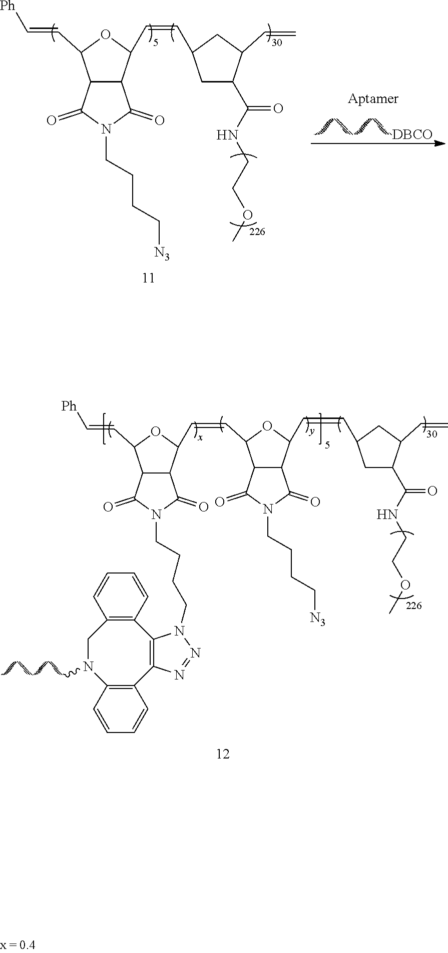

Synthesis of PSP pacDNA

The PSP backbone was prepared by solid-phase synthesis using a custom phosphoramidite. To graft the PEG side chains, 100 nmol of the PSP backbone polymer and N-hydroxysuccinimide (NHS)-terminated 10 kDa mPEG (30 mg PEG, 1:1 amine:PEG) were dissolved in 1 mL of phosphate buffered saline (PBS, pH 7.4). The mixture was shaken at 4° C. overnight and lyophilized to give a white powder, which was then redissolved in 1 mL anhydrous DMF containing 42 μL triethylamine. To this mixture, 30 mg of NHS-terminated PEG (dissolved in 1 mL DMF) was added in 5 aliquots (12 h between each aliquot), and the mixture was gently shaken at room temperature for 12 h after the last aliquot before being dried in vacuo. The PSP pacDNA was isolated from free 10 kDa PEG by fractionation using aqueous GPC. Note on scalability: the overall synthesis yield of the PSP pacDNA is similar to that of the free oligonucleotide plus ˜30 more synthesis cycles (the serinol phosphoramidite has high incorporation yields comparable to that of normal nucleotide phosphoramidites). The conversion from the PSP backbone into the bottlebrush polymer is close to 100% isolation yield as an excess of PEG is being used. Given existing mature technology for size exclusion-based purification, the PSP pacDNA synthesis should be similarly scalable as free oligonucleotides.

### Quantification of Amine Groups Using TNBSA Assay

To determine the number of amine groups before and after PEGylation, samples were dissolved in 100 μL of 0.1M sodium bicarbonate solution (pH 8.5). Then, 50 μL of 0.01% (w/v) 2,4,6-trinitrobenzene sulfonic acid (TNBSA) in 0.1 M sodium bicarbonate solution was added to each sample. For blank control, 50 μL of 0.1 M sodium bicarbonate solution without TNBSA was added. Next, the mixtures were incubated at 37° C. for 2 h. Next, 50 μL of 10% sodium dodecyl sulfate (SDS) solution and 25 μL of 1 N HCl were added to quench the reaction. Samples were then placed in a plate reader and absorbance at 335 nm was measured. The amine concentrations were calculated using a calibration curve established with a glycine standard.

### Hybridization Kinetics

Samples were dissolved in PBS (pH 7.4) at a final DNA concentration of 100 nM. A total of 1 mL solution for each sample was transferred to a quartz cuvette. Dabcyl-labeled complementary strand or dummy strand (2 equiv.) in 2 μL PBS solution were added into the cuvette and rapidly mixed with a pipette. The fluorescence of the solution (ex=640 nm, em=670 nm) was continuously monitored every 3 seconds for 30 min. The endpoint was determined by adding a large excess (10 equiv.) of the complementary dabcyl strand to the mixture. The kinetics plots were normalized to the endpoint determined for each sample, and all measurements were repeated 3×.

### Coarse-Grained Molecular Dynamics Simulations

An all-atom structure of the polymer biohybrid with the PSP backbone was mapped to coarse-grained (CG) beads according to functional groups that best match the bead types in the MARTINI force field (2-5 atoms per bead). The CG parameters for the polymer backbone and linkers were extracted from a molecular dynamics (MD) trajectory of an atomistic simulation of a three-repeating unit model molecule based on the OPLS-AA force field. The coarse-grained structure was solvated in a CG water box. Sodium ions were added to ensure the system is neutral in charge. The solvated system underwent energy minimization, followed by 50 ns of equilibration and 10 ns of production MD simulation (step size: 4 fs; NPT ensemble) using GROMACS 2021.3 with the velocity rescale thermostat and the Parrinello-Rahman barostat under 300 K and 1 bar.

### Nuclease Degradation Kinetics

Samples were each mixed with their complementary dabcyl-labeled DNA (2 equiv.) in PBS. The solutions were heated to 95° C. for 5 min and cooled down to room temperature, then shaken overnight. Next, 100 μL of each sample was withdrawn and diluted to 1 mL (100 nM) with assay buffer (10 mM Tris-HCl, 2.5 mM MgCl2, and 0.5 mM CaCl2), pH 7.5). The mixture was transferred to a quartz cuvette which was mounted on a fluorimeter. DNase I was added and rapidly mixed to give a final concentration of 0.6 unit/mL. The fluorescence of the samples (ex=640 nm, em=670 nm) was measured immediately and every 3 seconds for 2 h. The endpoint was determined by adding a large excess of DNase I (5 units/mL) to the solution followed by incubation for 2 h.

### Cell Culture

NCI-H358 and SKBR3 cells were cultured in RPMI 1640 media supplemented with 10% fetal bovine serum (FBS) and 1% antibiotics. Hep3B cells were cultured in DMEM media supplemented with 10% FBS and 1% antibiotics. HUVEC cells were cultured in endothelial cell basal medium 2 (PromoCell, Germany) and supplemented with SupplementPack endothelial cell GM2 (PromoCell, Germany). Cell culture flasks and well plates for HUVEC were coated with 0.1% gelatin overnight before use. All cells were cultured at 37° C. in a humidified atmosphere containing 5% CO₂.

### Cellular Uptake

Cellular uptake of samples and controls was evaluated using flow cytometry. Cells were seeded in 24-well plates at a density of 2.0×10⁵ cells per well in 1 mL full growth media and cultured for 24 h at 37° C. with 5% CO₂. After washing by PBS 1×, Cy3-labeled samples and controls (250 nM-5 μM equiv. of DNA) dissolved in serum-free culture media (400 μL) was added, and cells were further incubated at 37° C. for 4˜6 h. Next, cells were washed with PBS 2× and treated with trypsin (60 μL per well). Thereafter, 1 mL of PBS was added to each culture well to suspend the cells. Cells were then analyzed on an Attune™ NxT flow cytometer (Invitrogen, MA). Data for 1.0×10⁴ gated events were collected.

### Cytotoxicity Assay

The cytotoxicity of free HD1 and PSP pacHD1 was evaluated using the MTT (dimethylthiazol-diphenyltetrazolium bromide) colorimetric assay for NCI-H358 cells. Briefly, 8×10³ cells were seeded into 96-well plates in 200 μL RPMI media per well and were cultured for 24 h. The cells were then treated with HD1 and PSP pacHD1 at varying concentrations of oligonucleotides (0.1 through 5 μM; DNA basis). Cells treated with vehicle (PBS) were set as a negative control. After 48 h of incubation, 20 μL of 5 mg/mL MTT stock solution in PBS was added to each well. The cells were incubated for another 4 h, and the media containing unreacted MTT was removed carefully. The resulting blue formazan crystals were dissolved in DMSO (200 μL per well), and the absorbances (560 nm) were measured on a BioTek® Synergy™ Neo2 Multi-Mode microplate reader (BioTek Inc., VT, USA).

### Animals

Animals were housed and cared at Northeastern University animal facilities. Animal protocols (protocol number 19-0625R) were approved by the Institutional Animal Care and Use Committee of Northeastern University. Animal experiments and operations were conducted in accordance with the approved guidelines.

### Plasma Pharmacokinetics

8˜12-week-old female C57BL/6 mice were purchased from Charles River (MA, USA). Mice were randomly divided into 9 groups (n=4). Samples were intravenously administered via the tail vein at equal DNA concentrations (500 nmol/kg). Blood samples (25 μL) were collected from the submandibular vein at varying time points (30 min, 2 h, 4 h, 10 h, 24 h, 48 h and 72 h) using BD Vacutainer blood collection tubes with sodium heparin. Heparinized plasma samples were obtained by centrifugation at 3000 rpm for 20 min, and then aliquoted into a 96-well plate. The fluorescence intensities were measured on a plate reader. The amounts of agents in the blood samples were estimated using standard curves established for each sample prepared in freshly collected plasma.

### Biodistribution

8˜10-week-old female athymic mice and SKH1-Elite mice were purchased from Charles River (MA, USA). Cy5-labeled samples (10 nmol in 200 μL PBS) were injected into mice through the tail vein. Fluorescent images were collected at 1, 4, 8, 24 h and daily thereafter using an IVIS Lumina II imaging system (Caliper Life Sciences Inc., MA, USA). At predetermined time points, mice were euthanized using CO₂. Major organs (heart, liver, spleen, lung, kidney, brain, skin) were dissected and rinsed with PBS for biodistribution analysis.

### Microscale Thermophoresis

Microscale thermophoresis (MST) binding measurements were carried out with 10 nM Cy5-labeled human alpha thrombin as target. On average there were 2.5 Cy5 dyes per thrombin protein. Samples and controls were dissolved in binding buffer (20 mM HEPES, pH 7.4, 150 mM NaCl, 2 mM CaCl₂), and 0.05% TWEEN) at 10 μM as stock solutions. Then, 10 μL of each sample was serial-diluted (halving concentration each time) for a total of 16 dilutions, and each dilution was mixed with 10 μL of 20 nM Cy5-labeled human alpha thrombin solution. The mixtures were transferred to Monolith NT.115 standard capillaries and analyzed on a Monolith NT.115 instrument (NanoTemper Technologies, Munich, Germany) at medium MST power and 20% excitation power. Data were analyzed using MO. Affinity Analysis software (version 2.3, NanoTemper Technologies) and MST-on time was set at 1.5 s.

### Plasma Clotting Assays Using Human and Mouse Plasma

Prothrombin time (PT) and activated partial thromboplastin time (aPTT) assays were performed on a model BFT-2 coagulometer (Siemens, USA). Whole blood from C57BL/6 mice was collected in a tube containing sodium citrate solution (3.2% w/v) to a ratio of ˜9:1 (blood:citrate solution). Plasma was collected after centrifuging at 1500 g for 15 min. For PT, normal human plasma or mouse plasma (50 μL) were mixed with 5 μL of samples or controls to give a final DNA concentration of 5 μM. To test antidote efficacy, 5 μL of antidote (10 equiv. of complementary DNA) was added to the plasma. The samples were incubated at 37° C. for 5 min, to which 100 μL of thromboplastin-D (ThermoFisher, MA, USA) was added to initiate the coagulation. The time until clot formation was automatically recorded by the coagulometer. For aPTT, normal human plasma or mouse plasma (50 μL) was mixed with 5 μL of samples and controls to give a final DNA concentration of 5 μM. To test antidote efficacy, 5 μL of antidote (10 equiv. of complementary DNA) was added to the plasma. The samples were then incubated with 50 μL of aPTT-XL (ThermoFisher, MA, USA) at 37° C. for 5 min before 50 μL of CaCl₂ (0.025 M) was added to initiate the coagulation. The time until clot formation was automatically recorded by the coagulometer.

### In Vivo Blood Clotting

8˜10-week-old female C57BL/6 mice (Charles River, MA, USA) were randomly divided into 8 groups (n=5). Mice were administrated of samples and controls (15 nmol; DNA basis) through the tail vein. Blood samples (180 μL) collected at predetermined time points were mixed with 20 μL of 3.2% sodium citrate buffer for 5 min. Whole blood was centrifuged at 1500 g for 15 min at 4° C. For groups that test antidote efficacy, mice were injected with an LNA antidote (150 nmol) 20 min after the initial injection of PSP pacHD1, and blood was collected 30 min after the injection of PSP pacHD1. The collected plasma samples (50 μL) were analyzed by PT and aPTT assays.

### Murine Tail Transection Bleeding

8˜10-week-old female C57BL/6 mice (Charles River, MA, USA) were divided into 4 groups (n=5). Mice were injected with 200 μL of samples and controls (15 nmol; DNA basis) via the tail vein. After 5 min, the distal 1 mm of the tail was amputated. For groups testing antidote efficacy, 200 μL of an LNA antidote (150 nmol) was administered by tail vein injection immediately after the injection of the PSP pacHD1, and the clipping of the distal 1 mm of the tail was performed 10 min after the injection of the PSP pacHD1. Blood from the tail wound was collected for 15 min after tail transection, and the amount of blood loss was measured using the protocol described in Fay, William P., et al. “Vitronectin inhibits the thrombotic response to arterial injury in mice.” Blood, The Journal of the American Society of Hematology 93.6 (1999): 1825-1830 (the contents of which are herein incorporated by reference in their entirety).

### Innate Immune System Activation

To evaluate potential activation of the innate immune system to systemically delivered PSP pacDNA, immunocompetent C57BL/6 mice (n=4) were injected i.v. with samples and controls at an equal DNA concentration (0.75 μmol/kg). Lipopolysaccharide (LPS, 15 μg per animal) was used as a positive control. Serum samples were collected 2 h post injection and processed to measure complement C3 and representative cytokines (IL-6, IL-12 [p70], and TNF-α) using enzyme-linked immunosorbent assay (ELISA) kits according to the manufacturer's protocol (Mouse Complement C3 ELISA Kit, Abcam, Inc., MA, USA; Mouse IL-6, IL-12 and TNF-α ELISA Kits, R&D Systems, Inc., MN, USA).

### REFERENCES

Abraham, Mark James, et al. “GROMACS: High performance molecular simulations through multi-level parallelism from laptops to supercomputers.” SoftwareX 1 (2015): 19-25.
Akyol, Engin, and Andrew Lotery. “Gene, cell and antibody-based therapies for the treatment of age-related macular degeneration.” Biologics: Targets & Therapy 14 (2020): 83.
Andreeßen, Bjorn, and Alexander Steinbüchel. “Serinol: small molecule-big impact.” AMB Express 1.1 (2011): 1-6.
Aurup, Helle, David M. Williams, and Fritz Eckstein. “2′-Fluoro and 2-amino-2′-deoxynucleoside 5′-triphosphates as substrates for T7 RNA polymerase.” Biochemistry 31.40 (1992): 9636-9641.
Bock, Louis C., et al. “Selection of single-stranded DNA molecules that bind and inhibit human thrombin.” Nature 355.6360 (1992): 564-566.
Bussi, Giovanni, Davide Donadio, and Michele Parrinello. “Canonical sampling through velocity rescaling.” The Journal of Chemical Physics 126.1 (2007): 014101.
Charles, Laurence, and Jean-Frangois Lutz. “Design of abiological digital poly (phosphodiester)s.” Accounts of Chemical Research 54.7 (2021): 1791-1800.
Derszniak, Katarzyna, et al. “Comparison of effects of anti-thrombin aptamers HD1 and HD22 on aggregation of human platelets, thrombin generation, fibrin formation, and thrombus formation under flow conditions.” Frontiers in Pharmacology 10 (2019): 68.
Dodda, Leela S., et al. “LigParGen web server: an automatic OPLS-AA parameter generator for organic ligands.” Nucleic Acids Research 45.W1 (2017): W331-W336.
Dunn, Matthew R., Randi M. Jimenez, and John C. Chaput. “Analysis of aptamer discovery and technology.” Nature Reviews Chemistry 1.10 (2017): 1-16.
EDER, PAUL S., et al. “Substrate specificity and kinetics of degradation of antisense oligonucleotides by a 3′ exonuclease in plasma.” Antisense Research and Development 1.2 (1991): 141-151.
Ellington, Andrew D., and Jack W. Szostak. “In vitro selection of RNA molecules that bind specific ligands.” Nature 346.6287 (1990): 818-822.
Fay, William P., et al. “Vitronectin inhibits the thrombotic response to arterial injury in mice.” Blood, The Journal of the American Society of Hematology 93.6 (1999): 1825-1830.
Geng, Zhongmin, et al. “Enhancing anti-PD-1 Immunotherapy by Nanomicelles Self-Assembled from Multivalent Aptamer Drug Conjugates.” Angewandte Chemie 133.28 (2021): 15587-15593.
Grunewald, Fabian, et al. “Transferable MARTINI model of poly (ethylene oxide).” The Journal of Physical Chemistry B 122.29 (2018): 7436-7449.
Hakem, Ilhem F., Jyotsana Lal, and Michael R. Bockstaller. “Binding of monovalent ions to PEO in solution: Relevant parameters and structural transitions.” Macromolecules 37.22 (2004): 8431-8440.
Hong, Shanni, et al. “A photo-regulated aptamer sensor for spatiotemporally controlled monitoring of ATP in the mitochondria of living cells.” Chemical Science 11.3 (2020): 713-720.
Jia, Fei, et al. “Effect of PEG architecture on the hybridization thermodynamics and protein accessibility of PEGylated oligonucleotides.” Angewandte Chemie 129.5 (2017): 1259-1263.
Jorgensen, William L., David S. Maxwell, and Julian Tirado-Rives. “Development and testing of the OPLS all-atom force field on conformational energetics and properties of organic liquids.” Journal of the American Chemical Society 118.45 (1996): 11225-11236.
Juliano, R. L., et al. “Cellular uptake and intracellular trafficking of oligonucleotides: implications for oligonucleotide pharmacology.” Nucleic Acid Therapeutics 24.2 (2014): 101-113.
Kashida, Hiromu, et al. “Control of the chirality and helicity of oligomers of serinol nucleic acid (SNA) by sequence design.” Angewandte Chemie 123.6 (2011): 1321-1324.
Keefe, Anthony D., Supriya Pai, and Andrew Ellington. “Aptamers as therapeutics.” Nature Reviews Drug Discovery 9.7 (2010): 537-550.
KluBmann, Sven, et al. “Mirror-image RNA that binds D-adenosine.” Nature Biotechnology 14.9 (1996): 1112-1115.
Kovacevic, Katarina D., James C. Gilbert, and Bernd Jilma. “Pharmacokinetics, pharmacodynamics and safety of aptamers.” Advanced Drug Delivery Reviews 134 (2018): 36-50.
Kretz, Colin A., et al. “HD1, a thrombin-directed aptamer, binds exosite 1 on prothrombin with high affinity and inhibits its activation by prothrombinase.” Journal of Biological Chemistry 281.49 (2006): 37477-37485.
Lacroix, Aurelie, and Hanadi F. Sleiman. “DNA nanostructures: current challenges and opportunities for cellular delivery.” ACS Nano 15.3 (2021): 3631-3645.
Lao, Yeh-Hsing, Kyle K L Phua, and Kam W. Leong. “Aptamer nanomedicine for cancer therapeutics: barriers and potential for translation.” ACS Nano 9.3 (2015): 2235-2254.
Love, Jennifer A., et al. “A practical and highly active ruthenium-based catalyst that effects the cross metathesis of acrylonitrile.” Angewandte Chemie 114.21 (2002): 4207-4209.
Lu, Xueguang, et al. “Effective antisense gene regulation via noncationic, polyethylene glycol brushes.” Journal of the American Chemical Society 138.29 (2016): 9097-9100.
Lu, Xueguang, et al. “Effective antisense gene regulation via noncationic, polyethylene glycol brushes.” Journal of the American Chemical Society 138.29 (2016): 9097-9100.
Lu, Xueguang, et al. “Providing oligonucleotides with steric selectivity by brush-polymer-assisted compaction.” Journal of the American Chemical Society 137.39 (2015): 12466-12469.
Magarkar, Aniket, et al. “Molecular dynamics simulation of PEGylated bilayer interacting with salt ions: a model of the liposome surface in the bloodstream.” The Journal of Physical Chemistry B 116.14 (2012): 4212-4219.
Marrink, Siewert J., et al. “The MARTINI force field: coarse grained model for biomolecular simulations.” The Journal of Physical Chemistry B 111.27 (2007): 7812-7824.
Nelson, Paul S., Hugh Mackie, and Andrew Murphy. “Novel Reagents Utilizing A Serinol Scaffold For Labeling Synthetic Oligonucleotides.” U.S. patent application Ser. No. 12/891,888.
Ng, Eugene W M, et al. “Pegaptanib, a targeted anti-VEGF aptamer for ocular vascular disease.” Nature Reviews Drug Discovery 5.2 (2006): 123-132.
Ni, Shuaijian, et al. “Recent progress in aptamer discoveries and modifications for therapeutic applications.” ACS Applied Materials & Interfaces 13.8 (2020): 9500-9519.
Paborsky, Lisa R., et al. “The single-stranded DNA aptamer-binding site of human thrombin.” Journal of Biological Chemistry 268.28 (1993): 20808-20811.
Parrinello, Michele, and Aneesur Rahman. “Polymorphic transitions in single crystals: A new molecular dynamics method.” Journal of Applied Physics 52.12 (1981): 7182-7190.
Pearce, Amanda K., and Rachel K. O'Reilly. “Polymers for biomedical applications: the importance of hydrophobicity in directing biological interactions and application efficacy.” Biomacromolecules 22.11 (2021): 4459-4469.
Pontrello, Jason K., et al. “Solid-phase synthesis of polymers using the ring-opening metathesis polymerization.” Journal of the American Chemical Society 127.42 (2005): 14536-14537.
Riccardi, Claudia, et al. “G-quadruplex-based aptamers targeting human thrombin: Discovery, chemical modifications and antithrombotic effects.” Pharmacology & Therapeutics 217 (2021): 107649.
Roloff, Alexander, et al. “Micellar thrombin-binding aptamers: reversible nanoscale anticoagulants.” Journal of the American Chemical Society 139.46 (2017): 16442-16445.
Rusconi, Christopher P., et al. “Antidote-mediated control of an anticoagulant aptamer in vivo.” Nature Biotechnology 22.11 (2004): 1423-1428.
Rusconi, Christopher P., et al. “RNA aptamers as reversible antagonists of coagulation factor IXa.” Nature 419.6902 (2002): 90-94.
Schrlau, Michael G., Nae J. Dun, and Haim H. Bau. “Cell electrophysiology with carbon nanopipettes.” ACS Nano 3.3 (2009): 563-568.
Su, Chan-Fei, et al. “Nanoparticles of various degrees of hydrophobicity interacting with lipid membranes.” The Journal of Physical Chemistry Letters 8.17 (2017): 4069-4076.
Sykes, Edward A., et al. “Nanoparticle exposure in animals can be visualized in the skin and analysed via skin biopsy.” Nature Communications 5.1 (2014): 1-8.
Tao, Xiaoqi, et al. “Conjugation of antibodies and aptamers on nanozymes for developing biosensors.” Biosensors and Bioelectronics 168 (2020): 112537.
Tuerk, Craig, and Larry Gold. “Systematic evolution of ligands by exponential enrichment: RNA ligands to bacteriophage T4 DNA polymerase.” Science 249.4968 (1990): 505-510.
Uusitalo, Jaakko J., et al. “Martini coarse-grained force field: extension to DNA.” Journal of Chemical Theory and Computation 11.8 (2015): 3932-3945.
Vater, Axel, and Sven Klussmann. “Turning mirror-image oligonucleotides into drugs: the evolution of Spiegelmer® therapeutics.” Drug Discovery Today 20.1 (2015): 147-155.
Wang, Dali, et al. “Bottlebrush-architectured poly (ethylene glycol) as an efficient vector for RNA interference in vivo.” Science Advances 5.2 (2019): eaav9322.
Wang, Dali, et al. “Targeting oncogenic KRAS with molecular brush-conjugated antisense oligonucleotides.” Proceedings of the National Academy of Sciences 119.29 (2022): e2113180119.
Wu, Lingling, et al. “Aptamer-based detection of circulating targets for precision medicine.” Chemical Reviews 121.19 (2021): 12035-12105.
Yu, D., et al. “Cellular penetration and localization of polyethylene glycol.” Proceedings of the American Associationfor Cancer Research Vol. 45. 2004.
Zhao, Shuai, et al. “A DNA origami-based aptamer nanoarray for potent and reversible anticoagulation in hemodialysis.” Nature Communications 12.1 (2021): 1-10.
Zhou, Jiehua, and John Rossi. “Aptamers as targeted therapeutics: current potential and challenges.” Nature Reviews Drug Discovery 16.3 (2017): 181-202.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

1. A bottlebrush polymer-oligonucleotide conjugate (conjugate) comprising:
a) a sequence-defined polymer backbone comprising two or more monomers;
b) at least one side chain linked to at least one of the two or more monomers; and
c) at least one oligonucleotide linked to at least one of the two or more monomers.

2. The conjugate of claim 1, wherein:
a) at least one of the monomers is a phosphoramidite, protected amino acid, amino alcohol, amide, monomer comprising a serinol structure, monomer comprising a pentose structure, modified monomer, or any combination thereof;
b) the at least one side chain is a polysaccharide, a zwitterion polymer, or polyethylene glycol (PEG);
c) the at least one oligonucleotide is an aptamer, a single-stranded DNA, a double-stranded DNA, a single-stranded RNA, a double stranded RNA, a ribozyme, a DNAzyme, an antisense oligonucleotide, an exon-skipping oligonucleotide, an siRNA oligonucleotide, a triple helix forming oligonucleotide, or any combination thereof; or
d) any combination of the foregoing.

3. The conjugate of claim 2, wherein at least one of the monomers is a modified monomer and wherein the modified monomer is a monomer comprising a lipid tail, an aliphatic chain, a cholesterol molecule, a vitamin molecule, a sugar, an amino acid, a peptide, a targeting ligand, an ionizable group, or any combination thereof.

4. (canceled)

5. The conjugate of claim 1, wherein the arrangement of monomers comprising the sequence-defined polymer backbone is repeating, non-repeating, symmetrical, asymmetrical, arbitrary, or a combination thereof.

6. (canceled)

7. (canceled)

8. The conjugate of claim 1, wherein:
a) the sequence-defined polymer backbone comprises at least 5 monomers;
b) each of the monomers is covalently linked to at least 5 side chains; or
c) both of the foregoing.

9. (canceled)

10. The conjugate of claim 1, wherein each of the monomers comprising the sequence-defined polymer backbone has a defined number of possible side chain conjugation sites and wherein:
a) the percentage of side chains covalently linked to the side chain conjugation sites is at least 80%;
b) the defined number of conjugation sites per sequence-defined polymer is at least 5; or
c) both of the foregoing.

11. (canceled)

12. The conjugate of claim 1, wherein the at least one side chain is a polysaccharide, a zwitterion polymer, or polyethylene glycol (PEG).

13. The conjugate of claim 1, wherein the at least one oligonucleotide is linked to:
a) a terminal site of the backbone;
b) a non-terminal site of the backbone;
c) the backbone of the conjugate at a 3′ end of the at least one oligonucleotide, and the backbone of a second conjugate at a 5′ end of the at least one oligonucleotide;
d) the backbone via a cleavable bond; or
d) a combination of the foregoing.

14. (canceled)

15. The conjugate claim 1, wherein the at least one oligonucleotide:
a) is sufficiently complementary to a target polynucleotide to hybridize to the target polynucleotide or can bind to a non-nucleic acid target under predetermined conditions;
b) is chemically modified;
c) is between 8-40 nucleotides long;
d) further comprises a detectable label;
e) further comprises a linker used for conjugation to the polymer backbone; or
f) any combination of the foregoing.

16. The conjugate of claim 1, wherein the at least one oligonucleotide is an aptamer, a single-stranded DNA, a double-stranded DNA, a single-stranded RNA, a double stranded RNA, a ribozyme, a DNAzyme, an antisense oligonucleotide, an exon-skipping oligonucleotide, an siRNA oligonucleotide, a triple helix forming oligonucleotide, or any combination thereof.

17. (canceled)

18. (canceled)

19. (canceled)

20. (canceled)

21. (canceled)

22. (canceled)

23. The conjugate of claim 1, comprising two or more oligonucleotides, wherein the two or more oligonucleotides are identical, substantially identical, or substantially distinct.

24. (canceled)

25. The conjugate of claim 1, further comprising a pharmaceutically acceptable carrier.

26. A method of treating a disease or disorder, comprising administering to a subject in need thereof, a therapeutically effective amount of the bottlebrush polymer-oligonucleotide conjugate (conjugate) of claim 1.

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. The method of claim 26, wherein the conjugate is administered with an additional agent.

45. (canceled)

46. (canceled)

47. (canceled)

48. (canceled)

49. (canceled)

50. (canceled)

51. (canceled)

52. (canceled)

53. The method of claim 26, further comprising contacting a target polynucleotide with the conjugate, thereby modulating or altering the expression of a gene product encoded by the target polynucleotide.

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. The method of claim 53, wherein the target polynucleotide is a polynucleotide specific to a mammalian cancer cell, a mammalian non-cancer cell, a plant cell, a bacterium, or a virus.

80. (canceled)

81. The method of claim 53, wherein administration to a subject occurs in the absence of a transfection vector.

82. (canceled)

83. The method of claim 53, wherein the at least one oligonucleotide comprises a sufficiently complementary sequence to hybridize with the target polynucleotide and inhibit expression of the target polynucleotide, thereby inhibiting or reducing the expression of a gene product encoded by a target polynucleotide.

84. A method of making the bottlebrush polymer-oligonucleotide conjugate (conjugate) of claim 1, the method comprising:
a) polymerizing a sequence-defined polymer backbone comprising two or more monomers, wherein the polymerizing takes place via solid-phase synthesis or via synthesis in solution;
b) conjugating at least one side chain to at least one of the two or more monomers; and
c) conjugating at least one oligonucleotide to at least one of the two or more monomers.

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)

104. (canceled)

105. (canceled)

106. (canceled)

107. (canceled)

108. (canceled)

109. (canceled)

110. (canceled)

111. (canceled)

112. (canceled)

113. (canceled)

114. (canceled)

115. (canceled)

116. (canceled)

117. (canceled)

118. (canceled)

119. The method of claim 84, further comprising making a library of randomized backbones for use in making bottlebrush polymer-oligonucleotide conjugates, comprising:
polymerizing at least two sequence-defined polymer backbones, wherein each sequence-defined polymer backbone comprises two or more monomers, wherein the sequence of monomers comprising each backbone is different from each other backbone.

120. (canceled)

121. (canceled)

122. (canceled)
